(19) United States
(12) Patent Application Publication
(10) Pub. No.: US 2025/0222200 A1
(43) Pub. Date: Jul. 10, 2025

(54) CATHETER INSERTION DEVICE

(71) Applicant: Becton, Dickinson and Company, Franklin Lakes, NJ (US)

(72) Inventors: Jeffrey CHAGNON, Somerville, MA (US); Andrew J. RYAN, Boston, MA (US); Russell COLE, River Vale, NJ (US); Scott STEWART, Somerville, MA (US); Tieying JIANG, Shrewsbury, MA (US); Mark WOOD, Sterling, MA (US)

(73) Assignee: Becton, Dickinson and Company, Franklin Lakes, NJ (US)

(21) Appl. No.: 18/852,816

(22) PCT Filed: Mar. 30, 2023
(86) PCT No.: PCT/US2023/016911

§ 371 Date: Sep. 30, 2024

Related U.S. Application Data
(60) Provisional application No. 63/362,307, filed on Mar. 31, 2022.

(51) Int. Cl. A61M 5/158; A61M 5/14

(52) U.S. Cl. CPC A61M 5/158; A61M 5/1413; A61M 2005/1585; A61M 2205/0216

(57) ABSTRACT

A catheter insertion device is provided as an external applicator attachable to a medicine delivery device. The medicine delivery device has an internal module therein that comprises an insertion needle hub and needle, and a catheter hub and catheter. An external applicator comprises a drive and spring that drive a drive pin into the medicine delivery device to force the needle hub and catheter hub downward until the catheter hub locks in the downward position and the needle hub returns into the external applicator. An automatic release feature automatically disengaged the external applicator from the medicine delivery device when insertion is complete.

### FIELD OF THE INVENTION

The present invention relates generally to medical infusion systems, such as an insulin infusion device or insertion device, where simple, low-profile and low-part count manual insertion device is provided with a dual retraction spring configuration for automatic introducer needle retraction. The dual retraction spring configuration is implemented using multiple barrel-shaped guides and bosses in the insertion device housing which allows for much smaller retraction springs to be used than in a single-barrel configuration.

### BACKGROUND OF THE INVENTION

Diabetes is a group of diseases characterized by high levels of blood glucose resulting from the inability of diabetic patients to maintain proper levels of insulin production when required. Persons with diabetes will require some form of daily insulin therapy to maintain control of their glucose levels. Diabetes can be dangerous to the affected patient if it is not treated, and it can lead to serious health complications and premature death. However, such complications can be minimized by utilizing one or more treatment options to help control the diabetes and reduce the risk of complications.

The treatment options for diabetic patients include specialized diets, oral medications and/or insulin therapy. The main goal of diabetes treatment is to control the diabetic patient's blood glucose or sugar level. However, maintaining proper diabetes management may be complicated because it has to be balanced with the activities of the diabetic patient.

For the treatment of type 1 diabetes, there are two principal methods of daily insulin therapy. In the first method, diabetic patients use syringes or insulin pens to self-inject insulin when needed. This method requires a needle stick for each injection, and the diabetic patient may require three to four injections daily. The syringes and insulin pens that are used to inject insulin are relatively simple to use and cost effective.

Another effective method for insulin therapy and managing diabetes is infusion therapy or infusion pump therapy in which an insulin pump is used. The insulin pump can provide continuous infusion of insulin to a diabetic patient at varying rates in order to more closely match the functions and behavior of a properly operating pancreas of a non-diabetic person that produces the required insulin, and the insulin pump can help the diabetic patient maintain his/her blood glucose level within target ranges based on the diabetic patient's individual needs.

Infusion pump therapy requires an infusion cannula, typically in the form of an infusion needle or a flexible catheter, that pierces the diabetic patient's skin and through which, infusion of insulin takes place. Infusion pump therapy offers the advantages of continuous infusion of insulin, precision dosing, and programmable delivery schedules.

In infusion therapy, insulin doses are typically administered at a basal rate and in a bolus dose. When insulin is administered at a basal rate, insulin is delivered continuously over 24 hours in order to maintain the diabetic patient's blood glucose levels in a consistent range between meals and rest, typically at nighttime. Insulin pumps may also be capable of programming the basal rate of insulin to vary according to the different times of the day and night. In contrast, a bolus dose is typically administered when a diabetic patient consumes a meal, and generally provides a single additional insulin injection to balance the consumed carbohydrates. Insulin pumps may be configured to enable the diabetic patient to program the volume of the bolus dose in accordance with the size or type of the meal that is consumed by the diabetic patient. In addition, insulin pumps may also be configured to enable the diabetic patient to infuse a correctional or supplemental bolus dose of insulin to compensate for a low blood glucose level at the time when the diabetic patient is calculating the bolus dose for a particular meal that is to be consumed.

Insulin pumps advantageously deliver insulin over time rather than in single injections, typically resulting in less variation within the blood glucose range that is recommended. In addition, insulin pumps may reduce the number of needle sticks which the diabetic patient must endure, and improve diabetes management to enhance the diabetic patient's quality of life.

Typically, regardless of whether a diabetic patient uses multiple direct injections (MDIs) or a pump, the diabetic patient takes fasting blood glucose medication (FBGM) upon awakening from sleep, and also tests for glucose in the blood during or after each meal to determine whether a correction dose is required. In addition, the diabetic patient may test for glucose in the blood prior to sleeping to determine whether a correction dose is required, for instance, after eating a snack before sleeping.

To facilitate infusion therapy, there are generally two types of insulin pumps, namely, conventional pumps and patch pumps. Conventional pumps require the use of a disposable component, typically referred to as an infusion set, tubing set or pump set, which conveys the insulin from a reservoir within the pump into the skin of the user. The infusion set consists of a pump connector, a length of tubing, and a hub or base from which a cannula, in the form of a hollow metal infusion needle or flexible plastic catheter extends. The base typically has an adhesive that retains the base on the skin surface during use. The cannula can be inserted onto the skin manually or with the aid of a manual or automatic insertion device. The insertion device may be a separate unit required by the user.

Another type of insulin pump is a patch pump. Unlike a conventional infusion pump and infusion set combination, a patch pump is an integrated device that combines most or all of the fluidic components, including the fluid reservoir, pumping mechanism and mechanism for automatically inserting the cannula, in a single housing which is adhesively attached to an infusion site on the patient's skin, and does not require the use of a separate infusion or tubing set. A patch pump containing insulin adheres to the skin and delivers the insulin over a period of time via an integrated subcutaneous cannula. Some patch pumps may wirelessly communicate with a separate controller device (as in one device sold by Insulet Corporation under the brand name OmniPod®), while others are completely self-contained. Such devices are replaced on a frequent basis, such as every three days, when the insulin reservoir is exhausted or complications may otherwise occur, such as restriction in the cannula or the infusion site.

As patch pumps are designed to be a self-contained unit that is worn by the diabetic patient, it is preferable to be as small as possible so that it does not interfere with the activities of the user. Thus, in order to minimize discomfort to the user, it would be preferable to minimize the overall thickness of the patch pump. However, in order to minimize the thickness of the patch pump, its constituent parts should be reduced as much as possible. One such part is the insertion mechanism for automatically inserting the cannula into the user's skin.

In order to minimize the height of the insertion mechanism, some conventional insertion mechanisms are configured to insert the cannula at an acute angle from the surface of the skin, e.g. 30-45 degrees. However, it may be preferable to insert the cannula perpendicular or close to the perpendicular from the surface of the skin, since this would require the minimum length of cannula insertion. In other words, with the minimum length of cannula being inserted into the user's skin, the user can experience greater comfort and fewer complications, such as premature kinking of the cannula. But one problem with configuring the insertion mechanism to insert the cannula perpendicular to the surface of the skin is that this may increase the overall height of the insertion mechanism, and therefore of the patch pump itself.

Accordingly, a need exists for an improved insertion mechanism for use in a limited space environment, such as in the patch pump, that can cost-effectively insert a cannula vertically or close to perpendicularly into the surface of a user's skin, while minimizing or reducing its height, in order to reduce the overall height of the device the insertion mechanism is incorporated into, such as a patch pump.

### SUMMARY OF THE INVENTION

An object of the present invention is to substantially address the above and other concerns, and provide advanced, improved, and novel components and elements of an insertion device that facilitates insertion of the in-dwelling or soft catheter and retract the introducer needle, while reducing the number of components required for the construction and use of the insertion device.

Another object of the present invention is to provide a manual insertion device with at least automatic introducer needle retraction, such that the part count of the exemplary embodiments is lowered and which serves to keep part production costs low and simplify device assembly. Automatic retraction also simplifies the user interface by minimizing the number of user steps for activation. There is only one step for the user which is pushing the button.

Another object of the present invention is to provide a manual insertion device with at least automatic introducer needle retraction using a dual retraction spring configuration that is implemented using multiple barrel-shaped guides and bosses in the insertion device housing which allows for much smaller retraction springs to be used, such that the device is smaller and more compact.

Another object of the present invention is to provide a manual insertion device with at least automatic introducer needle retraction and activation button locking to provide needle shielding and maintain insertion of the catheter.

These and other objects are substantially achieved by providing an insertion device with a dual retraction spring configuration for automatic introducer needle retraction. The dual retraction spring configuration is implemented using multiple barrel-shaped guides and bosses in the insertion device housing which allows for much smaller retraction springs to be used than in a single-barrel configuration. A button of the insertion device is used to insert the introducer needle and catheter, and once the introducer needle and catheter have been fully inserted, a rotating engagement releases the dual retraction springs such that the introducer needle automatically retracts, leaving the catheter in the body of the user. An end of the introducer needle remains in the inserted catheter or wedge that holds the catheter and/or in the septum of the inserted catheter to provide an uninterrupted fluid path.

Additional and/or other aspects and advantages of the present invention will be set for in the description that follows, or will be apparent from the description, or may be learned by the practice of the invention. The present invention may comprise a method or apparatus or system having one or more of the above aspects, and/or one or more of the features and combinations thereof. The present invention may comprise one or more of the features and/or combinations of the above aspects as recited, for example, in the attached claims.

### BRIEF DESCRIPTION OF THE DRAWINGS

The various objects, advantages and novel features of the exemplary embodiments of the present invention will be more readily appreciated from the following detailed description when read in conjunction with the appended drawings, in which:

FIG. 1 is an isometric view of an exemplary insertion device in a pre-activation state in accordance with an embodiment of the present invention;

FIG. 2 is another isometric view of the insertion device of FIG. 1 in a pre-activation state in accordance with an embodiment of the present invention;

FIG. 3 is a view of the insertion device of FIG. 1 in a post-activation state in accordance with an embodiment of the present invention;

FIG. 4 is an exploded view of the insertion device of FIG. 1 in accordance with an embodiment of the present invention;

FIG. 5 is a sectional view of a catheter/septum subassembly of the insertion device of FIG. 1 in accordance with an embodiment of the present invention;

FIG. 6 is a view of an introducer needle subassembly, assembled from the top with plastic tubing, of the insertion device of FIG. 1 in accordance with an embodiment of the present invention;

FIG. 7 is a view of another introducer needle subassembly, assembled from the side with no plastic tubing, of the insertion device of FIG. 1 in accordance with an embodiment of the present invention;

FIG. 8 is a view of the assembly of the button subassembly of the insertion device of FIG. 1, including the catheter/septum subassembly of FIG. 5 and introducer needle subassembly of FIG. 6, in accordance with an embodiment of the present invention;

FIG. 9 is a view of the completed button subassembly of the insertion device of FIG. 1 in accordance with an embodiment of the present invention;

FIG. 10 is a view of the assembly of the button subassembly and springs into the housing of the insertion device of FIG. 1 and illustrating the use of temporary protective tubing on the catheter in accordance with an embodiment of the present invention;

FIG. 11 is a view of the partially complete assembly of the button subassembly and springs into the housing of the insertion device of FIG. 1 and illustrating the use of temporary protective tubing on the catheter in accordance with an embodiment of the present invention;

FIG. 12 is a view of the completed assembly of the insertion device of FIG. 1 wherein the base is omitted for illustration purposes in accordance with an embodiment of the present invention;

FIG. 13 is a sectional view of the insertion device of FIG. 1 in a pre-activation state in accordance with an embodiment of the present invention;

FIG. 14 is another sectional view of the insertion device of FIG. 1 in a pre-activation state in accordance with an embodiment of the present invention;

FIG. 15 is a sectional view of the insertion device of FIG. 1 in an intermediate activation state in accordance with an embodiment of the present invention:

FIG. 16 is a transparent view of the insertion device of FIG. 1 in the intermediate activation state illustrating a position of a radial operation pin within a helical pathway in accordance with an embodiment of the present invention;

FIG. 17 is a bottom view of the top housing of the insertion device of FIG. 1 illustrating a mating portion of the helical pathway surface for the radial operation pin of FIG. 16 in accordance with an embodiment of the present invention;

FIG. 18 is a view of the mechanism housing of the insertion device of FIG. 1 illustrating a mating portion of the helical pathway surface for the radial operation pin of FIG. 16 in accordance with an embodiment of the present invention;

FIG. 19 is a transparent view of the insertion device of FIG. 1 in an intermediate activation state illustrating a position of the radial operation pin in accordance with an embodiment of the present invention;

FIG. 20 is a transparent view of the insertion device of FIG. 1 at an activation state illustrating a position of the radial operation pin at full insertion in accordance with an embodiment of the present invention;

FIG. 21 is a transparent view of the insertion device of FIG. 1 at the post-activation state illustrating a position of the radial operation pin at full retraction in accordance with an embodiment of the present invention;

FIG. 22 is a sectional view of the insertion device of FIG. 1 illustrating a lock arm of the top housing in a post-activation state in accordance with an embodiment of the present invention;

FIG. 23 is a sectional view of the insertion device of FIG. 1 in a post-activation state in accordance with an embodiment of the present invention;

FIG. 24 is another exploded view of the insertion device of FIG. 1 in accordance with an embodiment of the present invention;

FIG. 25 is a view of another embodiment of the insertion mechanism created as a subassembly separate from the top housing or base in accordance with an embodiment of the present invention;

FIG. 26 is a view of another embodiment of the insertion mechanism created as a subassembly in the base in accordance with an embodiment of the present invention;

FIG. 27 is a sectional view of another embodiment of a lock arm in a pre-activation state in accordance with an embodiment of the present invention;

FIG. 28 is a sectional view of the lock arm of FIG. 27 in an intermediate activation state during insertion in accordance with an embodiment of the present invention shows the device;

FIG. 29 is a sectional view of the lock arm of FIG. 27 in a post-activation state in accordance with an embodiment of the present invention shows the device;

FIG. 30 is a perspective view of a patch pump incorporating a low-profile cannula insertion device, illustrated without a cover for clarity;

FIG. 31 is an exploded view of the various components of the patch pump of FIG. 30, illustrated with a cover;

FIG. 32 is a perspective view of an alternative design for a patch pump having a flexible reservoir, illustrated without a cover;

FIG. 33 is a patch-pump fluidic architecture and metering sub-system diagram of the patch pump of FIG. 32;

FIG. 34 is an isometric view of an alternate embodiment of the present disclosure;

FIGS. 35 and 36 illustrate an external applicator being assembled to a medicine delivery device according to an exemplary embodiment of the disclosure;

FIG. 37 is an exploded view of the internal module and external applicator subassemblies;

FIG. 38 is an assembled view of the internal module and external applicator subassemblies;

FIG. 39 is a cutaway view of opposite sides of the external applicator;

FIGS. 40A-40C illustrate the catheter insertion sequence;

FIGS. 41A-41B illustrate the release sequence of the external applicator from the medicine delivery device; and

FIG. 42 illustrates how flex arms of the internal module lock the catheter hub in the deployed position.

Throughout the drawings, like reference numerals will be understood to refer to like parts, components and structures.

### DETAILED DESCRIPTION OF THE EXEMPLARY EMBODIMENTS

The exemplary embodiments of the present invention described below provide novel means of providing one or more infusion device elements that are configured to insert catheter up to 8 mm into a skin surface, but embodiments are not limited thereto. The insertion device is configured to perform a manual insertion of the catheter which allows the insertion device to be smaller, simpler and cheaper than automatic or spring-assisted insertion devices.

Exemplary embodiments of the present invention described below, utilize a manual insertion device and include a dual retraction spring configuration for automatic introducer needle retraction that also allows for a very small device size. The dual retraction spring configuration is implemented using a plurality of cylindrical or barrel-shaped guides. In an exemplary embodiment, one barrel guides a button and catheter, and adjacent barrels house retraction springs, one on each side of the button and catheter. Having the springs in separate barrels allows for much smaller springs than a single-barrel configuration in which the spring is coaxial with the catheter. A single coaxial spring creates access to the button assembly since spring design limitations require the spring to extend nearly from the bottom of the housing to the top. Access is required for features like the locking arm and if the features are implemented inside the spring, the entire mechanism must grow to accommodate them increasing the mechanism foot print.

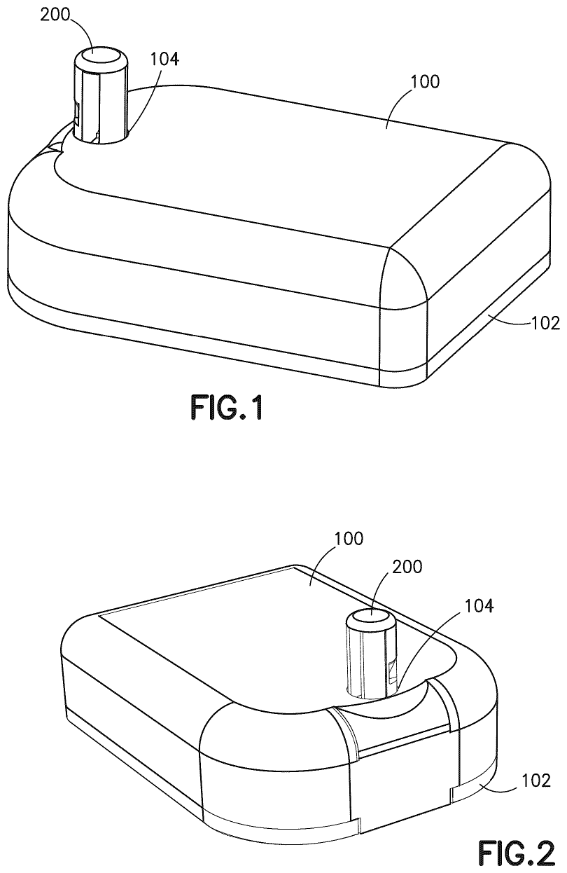

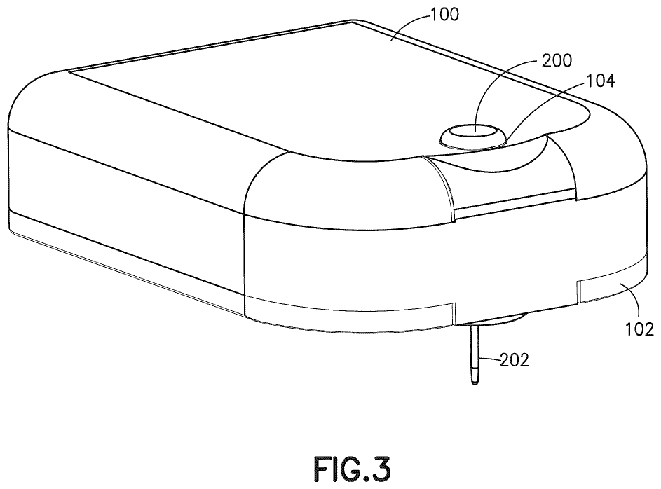

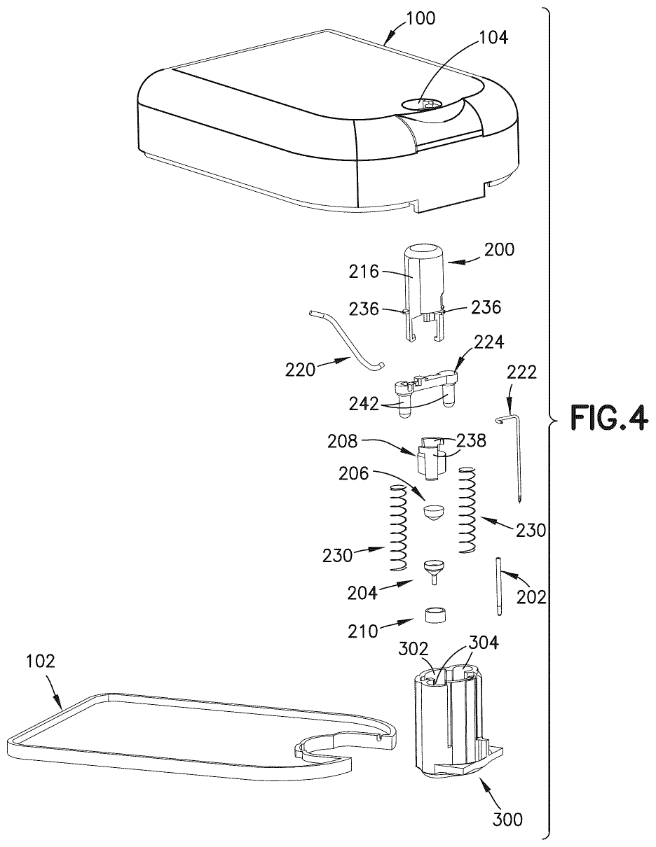

FIGS. 1 and 2 show the insertion device before use and FIG. 3 shows the device after deployment of the cannula. As shown in FIGS. 1-3, the insertion device includes a top housing 100 and a base 102. The top housing 100 is shown having an opening 104 through a top surface from which a user-accessible, and user-acutatable button 200 slidably extends. The content of the insertion device, including the mechanism housing 300, is shown in greater detail in FIG. 4. The top housing 100, button 200, and mechanism housing 300 can be manufactured from ABS, and the base 102 can be manufactured from PETG, but embodiments are not limited thereto.

As shown in FIG. 4, the exemplary insertion device is assembled by stacking together a number of subassemblies which are trapped between the top housing 100 and the mechanism housing 300. FIG. 4 is a view of the insertion device of FIG. 1 in accordance with an embodiment of the present invention. The subassemblies of FIG. 4 and discussed in greater detail below include a catheter septum subassembly, an introducer needle subassembly, and a button subassembly. Other features and functions of the insertion device that are well-known to those skilled in the art are omitted from the figures and discussion for clarity.

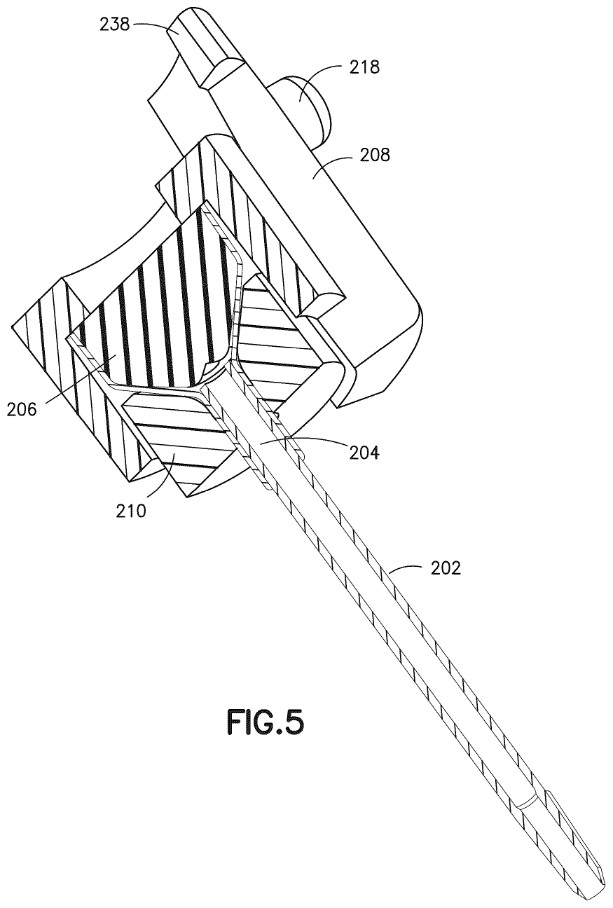

An exemplary catheter/septum subassembly is shown in FIG. 5. FIG. 5 is a sectional view of a catheter/septum subassembly of the insertion device of FIG. 1 in accordance with an embodiment of the present invention. As shown in FIG. 5, the catheter/septum subassembly is assembled by attaching a catheter 202 on a metal wedge 204, then inserting a septum 206 in the wedge and trapping it between a release collar 208 and a catheter wedge cap 210. The septum 206 is radially compressed by the wedge 204 and axially compressed by the release collar 208 to create a seal between the septum 206 and wedge 204. The catheter 202 can be a 24G plastic catheter manufactured using FEP, and the release collar 208 and catheter wedge cap 210 can be manufactured using PTEG, but embodiments are not limited thereto. The wedge 204 can be manufactured using 305 stainless steel, and the septum 206 can be manufactured using isoprene, but embodiments are not limited thereto.

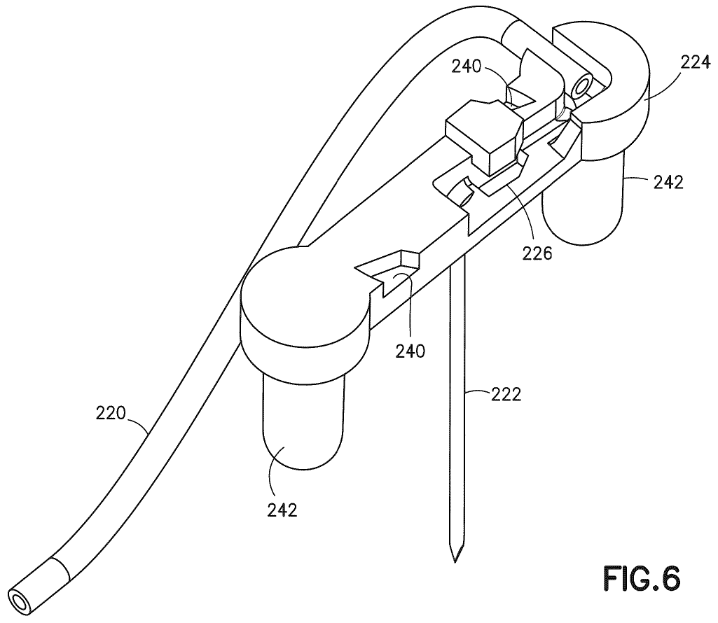

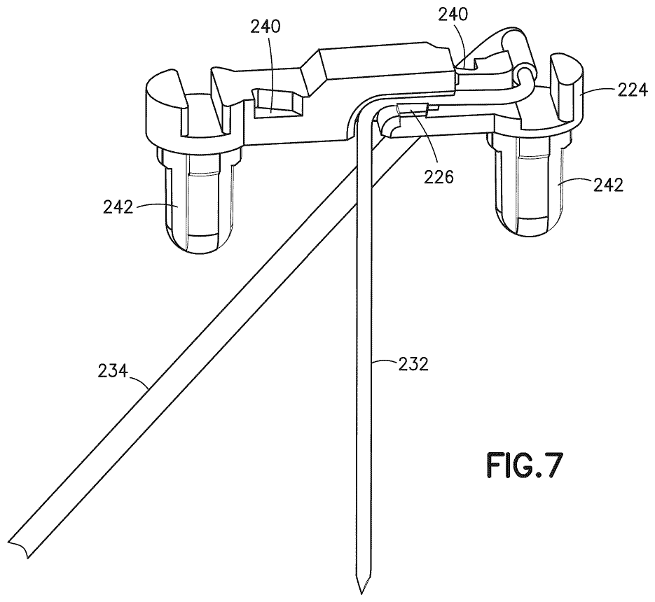

Exemplary introducer needle subassemblies are shown in FIGS. 6 and 7. FIG. 6 is a view of an introducer needle subassembly, assembled from the top with plastic tubing, and FIG. 7 is a view of another introducer needle subassembly, assembled from the side with no plastic tubing, of the insertion device of FIG. 1 in accordance with an embodiment of the present invention. The introducer needle subassembly of FIG. 6 and used in the following discussion is assembled by gluing or press-fitting tubing 220 on the non-patient end of the cannula or introducer needle 222, then placing the introducer needle through an introducer needle hub 224 and snapping it in place using any number of grooves, slots or detents 226 provided on a top surface of the introducer needle hub 224. The introducer needle 222 can be a hollow. 24G needle or cannula manufactured using 304 stainless steel, and the introducer needle hub 224 can be manufactured using PETG, but embodiments are not limited thereto.

An alternative embodiment of the introducer needle subassembly of FIG. 7 is assembled using an introducer needle 232 with a long proximal end 234 that connects directly to the pump or reservoir (not shown). Eliminating the flexible plastic tubing in this embodiment makes assembly of the insertion device easier and reduces the risks associated with attaching the two parts, but requires a large loop on the proximal end 234 of the cannula to reduce the force needed to bend the cannula during insertion and retraction.

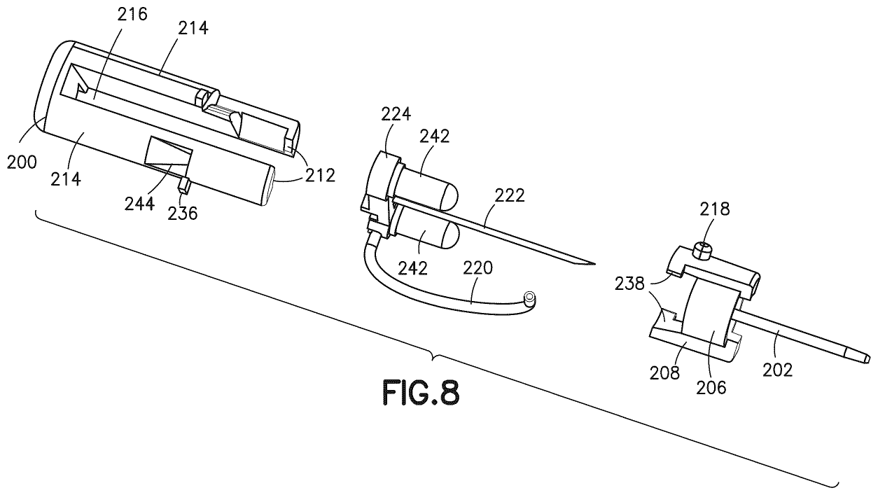

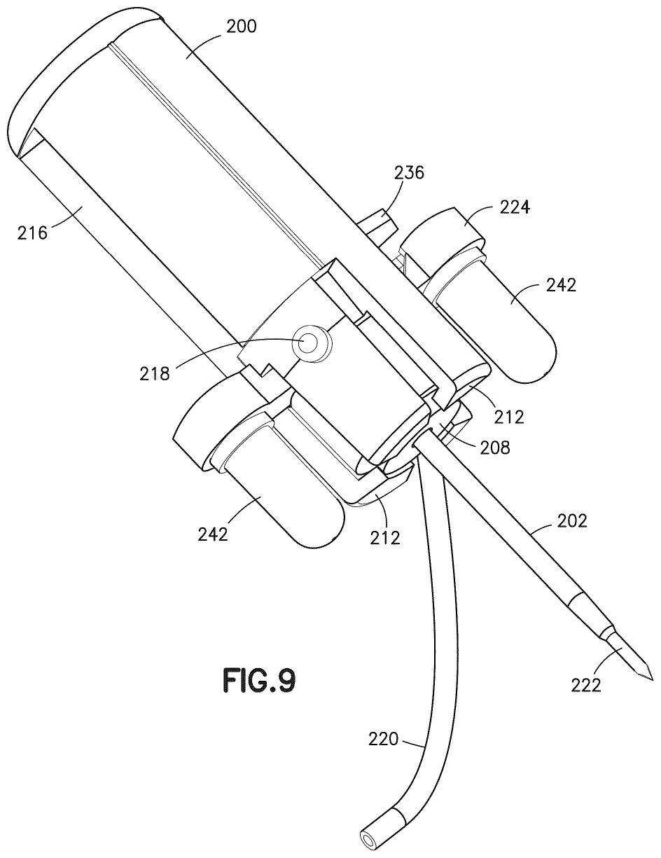

An exemplary button subassembly is shown in FIG. 8. FIG. 8 is a view of the assembly of the button subassembly of the insertion device of FIG. 1, including the catheter/septum subassembly and introducer needle subassembly, and FIG. 9 is a view of the completed button subassembly of the insertion device of FIG. 1 in accordance with an embodiment of the present invention. The button subassembly is built by combining the catheter/septum subassembly and introducer needle subassembly with the button 200. As described in greater detail below, once assembled, the introducer needle subassembly cannot be rotated in the button 200. The catheter/septum subassembly can be rotated in the button 200 and in doing so, can be rotated from a position secured with the introducer needle subassembly, to a position freed from the introducer needle subassembly.

Specifically, the button subassembly is built by inserting the introducer needle 222 of the introducer needle subassembly through the septum 206 and catheter 202 of the catheter/septum subassembly. The catheter/septum subassembly is then secured to the introducer needle subassembly by rotating the catheter/septum subassembly up to 20 degrees or more to lock the detents or teeth 238 on the release collar 208 into grooves or slots 240 on the top surface of the introducer needle hub 224, which couples the introducer needle bub 224 and catheter/septum subassembly. In this position the teeth 238 are locked over the top the introducer needle hub 224 so as the button 200 is pressed down, the introducer needle bub 224 also moves down. This results in the introducer needle 222 and catheter 202 being moved simultaneously for insertion into a user skin surface (not shown).

The button subassembly is then completed by snapping the release collar 208 into the button 200 to secure the introducer needle subassembly and the catheter/septum subassembly in place. To do so, the button 200 can include detents 212 on deflectable arms 214 to deflect and then capture therebetween the lower edge of the release collar 208 as shown in FIG. 9. Between the deflectable arms 214, slots 216 are provided in the button 200 to allow linear travel of the introducer needle hub 224 relative to the button 200, but prohibit rotational movement of the introducer needle hub relative to the button 200. The slots 216 in the button 200 also allow rotational movement of the radial operation pin 218 of the release collar 208 relative to the button 200 as described in greater detail below. In the exemplary embodiment, a substantially cylindrical-shaped pin 218 is shown on an outer circumference of the release collar 208. However, in this or other embodiments of the present invention, any detent or projection of the release collar which can operate with the helical pathway can be provided as the radial operation pin.

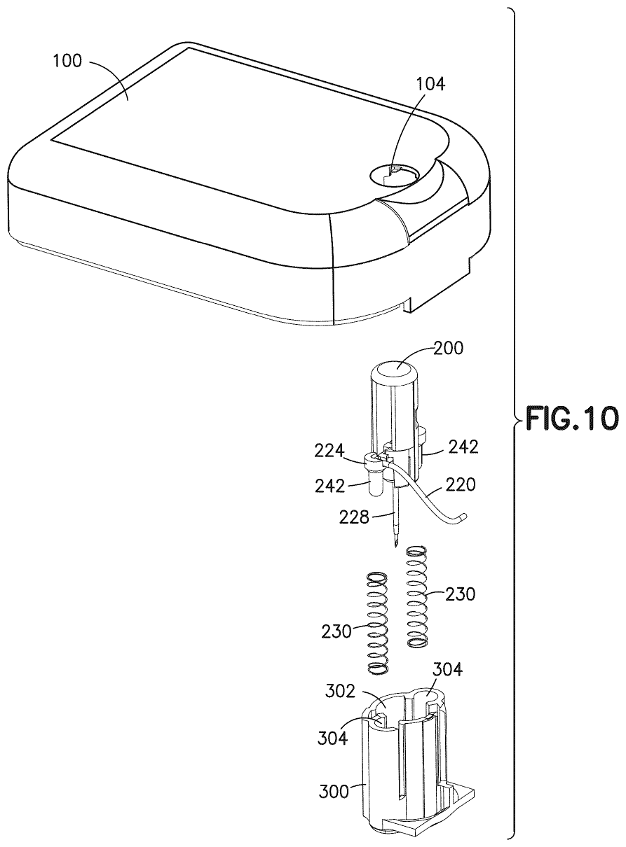

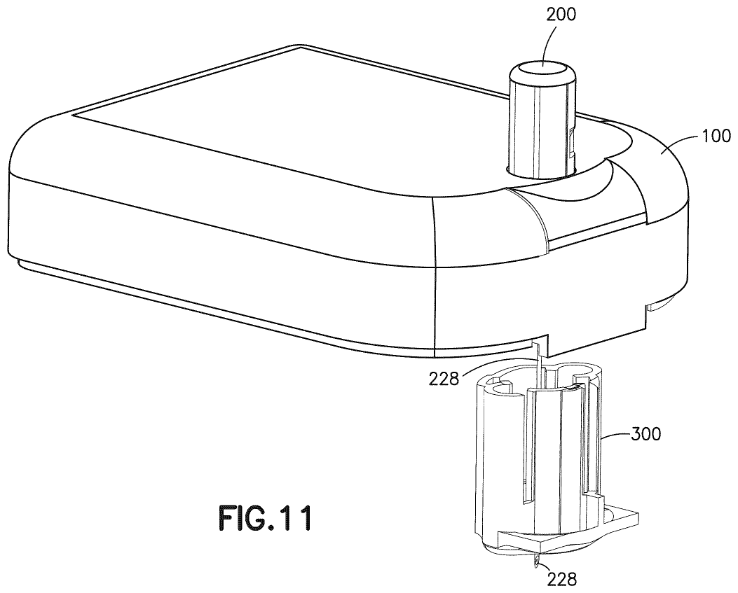

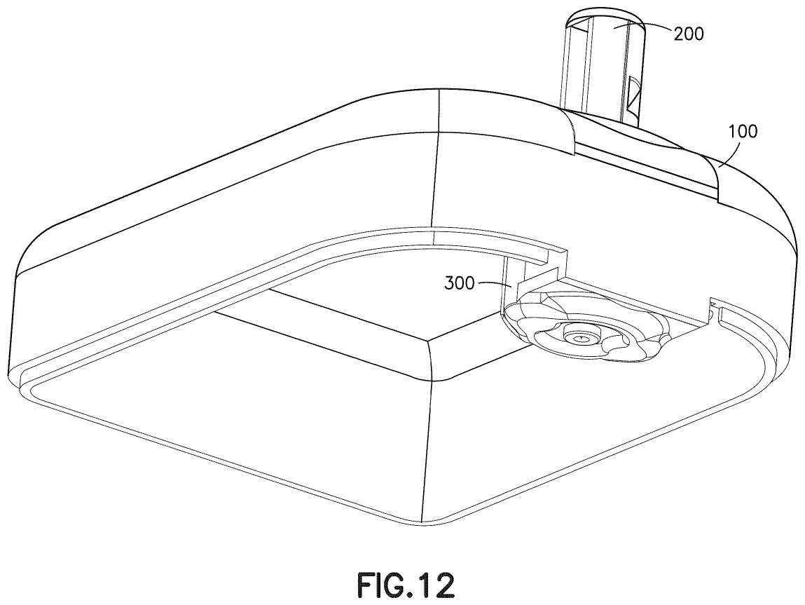

The button subassembly can then be assembled with the housing top 100 and mechanism housing 300. FIG. 10 is a view of the assembly of the button subassembly and springs into the housing of the insertion device of FIG. 1 and illustrating the use of temporary protective tubing on the catheter, and FIG. 11 is a view of the partially complete assembly of the button subassembly and springs into the housing of the insertion device of FIG. 1. FIG. 12 is a view of the completed assembly of the insertion device of FIG. 1 wherein the base is omitted for illustration purposes in accordance with an embodiment of the present invention.

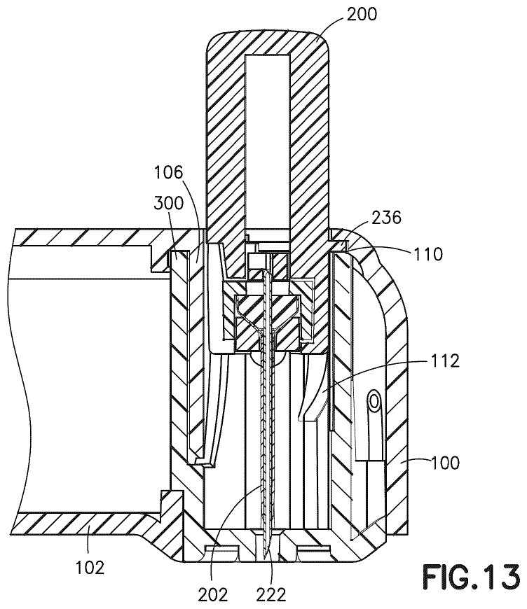

To complete assembly, the button 200 and assembly thereof is slidably assembled with a projection 106 extending from an inner surface of the top housing 100 as shown in greater detail in FIG. 13. FIG. 13 is a sectional view of the fully assembled insertion device of FIG. 1 in a pre-activation state in accordance with an embodiment of the present invention. A button lock arm 112 of the top housing 100 retains the button subassembly in place during the next assembly step which is placing the mechanism housing 300 into the top housing 100 thereby trapping the other subassemblies therein.

During the placement of the mechanism housing 300 into the top housing 100, a piece of temporary tubing 228 is placed over the catheter 202 and introducer needle 222 therein to both protect the needle tip and guide the catheter through the exit bole in the mechanism housing 300 during assembly. Retraction springs 230 are press fit onto the introducer needle hub 224 and the button subassembly is inserted through the hole 104 in the top housing 100 as shown in FIG. 11. The tubing or cannula 220 that connects to the reservoir or pump (not shown) is sealed in a receiving feature in the top housing. The springs 230 can be manufactured using stainless steel, but embodiments are not limited thereto.

The mechanism housing 300 is preferably comprised of three cylinders, guides or barrels, including a center barrel 302 that slidably receives and guides the button subassembly, and two barrels 304, one on each side of the center barrel 302 that constrain the springs 230. During assembly, the springs 230 are captured between bosses 242 of the introducer needle hub 224 and a bottom of the barrels 304 of the mechanism housing 300. In doing so, the springs 230 exert an expansion force between the introducer needle hub 224 and a bottom of the barrels 304 of the mechanism housing 300. In the exemplary embodiment, a plurality of springs 230 and adjacent barrels 304 are shown. However, in this or other embodiments of the present invention, a single spring and adjacent barrel can be provided in substantially the same manner, wherein the unused adjacent barrel can be left empty or can be omitted entirely. Still further, a single spring can be provided in the button top and extended during insertion that, upon completion, retracts to its natural state thereby retracting the introducer needle from the catheter.

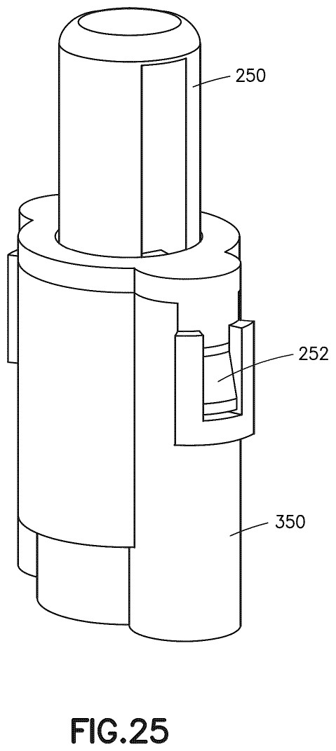

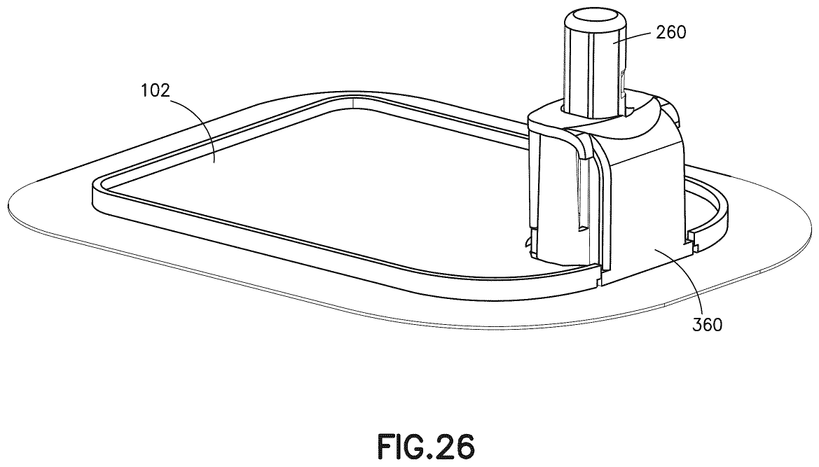

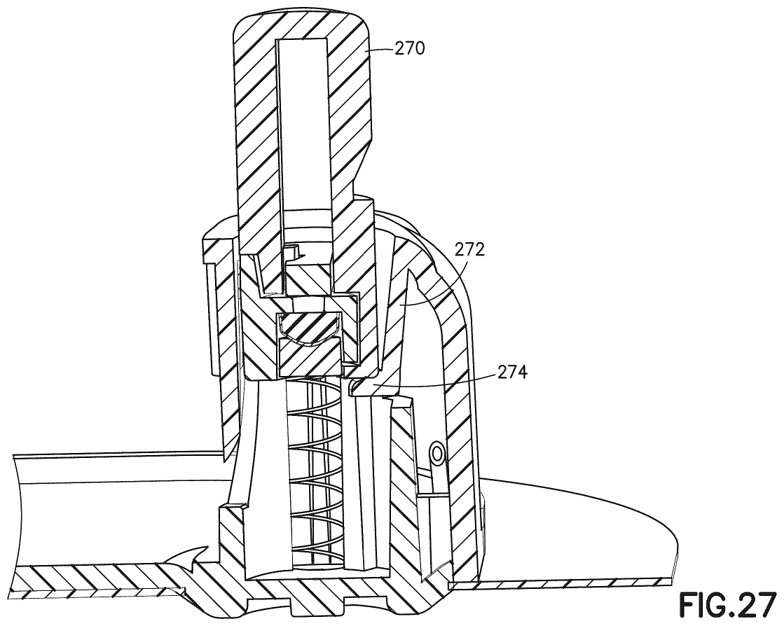

The rounded, bosses 242 are provided with a diameter and length to center and align the springs 230 during operation. The springs 230 can be partially preloaded during assembly of the insertion device, and the mechanism housing 300 can be laser welded or glued to the top housing 100. The bottom or base 102 can then be added. In doing so, the full and complete insertion mechanism subassembly can be placed onto the base 102 with all of the other components, as the last assembly step. Having the completed insertion mechanism subassembly allows for easy handling in production, as opposed to trapping all of the parts between the top and bottom housings. In an exemplary production, the mechanism housing 300 would be attached to the top housing 100 using snaps or adhesive (not shown) which holds together the mechanism. In yet two other exemplary embodiments described below in regard to FIGS. 25 and 26, similar subassembly concepts are used to make assembly manageable, but the subassembly is an independent unit in one embodiment and part of the base in the other.

In each embodiment, after finally assembly, the insertion device is hermetically sealed from the remainder of the device. That is, the mechanism housing 300 into which water from a shower or swimming is free to enter through the catheter exit hole or from the button hole in the housing top, is hermetically sealed with the laser welding or gluing step, thereby protecting the remaining content of the device housing 100, such as content of the electronic/pump compartments of the device.

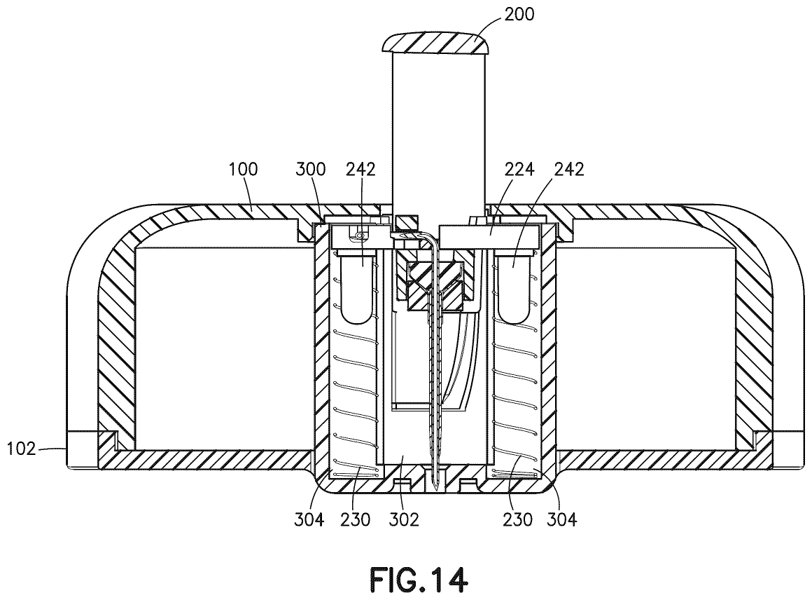

FIG. 13 is a sectional view of the fully assembled insertion device of FIG. 1 and FIG. 14 is another sectional view perpendicular to the view of FIG. 13 of the fully assembled insertion device of FIG. 1 in a pre-activation state in accordance with an embodiment of the present invention. As shown in FIG. 13, one or more breakable ribs 236 on the activation button 200 are captured by step detents 110 in the top housing 100 to hold the button 200 in the pre-activation position. A safety tab (not shown) could also be positioned in the button slot which would prevent accidental activation during shipping and handling of the device once it is removed from the packaging. The safety tab would be removed just prior to insertion.

To activate the device, the user pushes the button 200 into the top housing 100. Once the ribs 236 break or deformation force threshold is exceeded, the three ribs 236 yield and the button 200 abruptly moves downward inserting the introducer needle 222 and catheter 202, and loading the retraction springs 230. The springs 230 can be partially preloaded during assembly of the insertion device. The minimum break force of the breakable ribs 236 ensures that the user pushes hard enough to fully insert the catheter. Partial activation would result in the catheter not fully inserting, the introducer needle not retracting and the catheter not locking in the post activation position.

The release of the button 200 from the ribs 236 is configured to occur once a desired amount of activation force has been applied to the button 200. Since the button 200 is releasably held in the up and extended position by the engagement between the ribs 236 and the step detents 110, the force applied to the button 200 by the user steadily increases for some period of time prior to release. Upon sudden release, the force upon the button 200 has reached a desired value and therefore, the button 200 is accelerated downward due to the sudden freedom to travel and the desired force applied to the button at the time of release and maintained thereafter. Such release ensures that a desired amount of downward force, speed, smoothness and angle has been applied by the user. Such activation substantially eliminates variations in the user force applied, speed, smoothness and angle thereof, and reduces insertion failure and/or discomfort to the user.

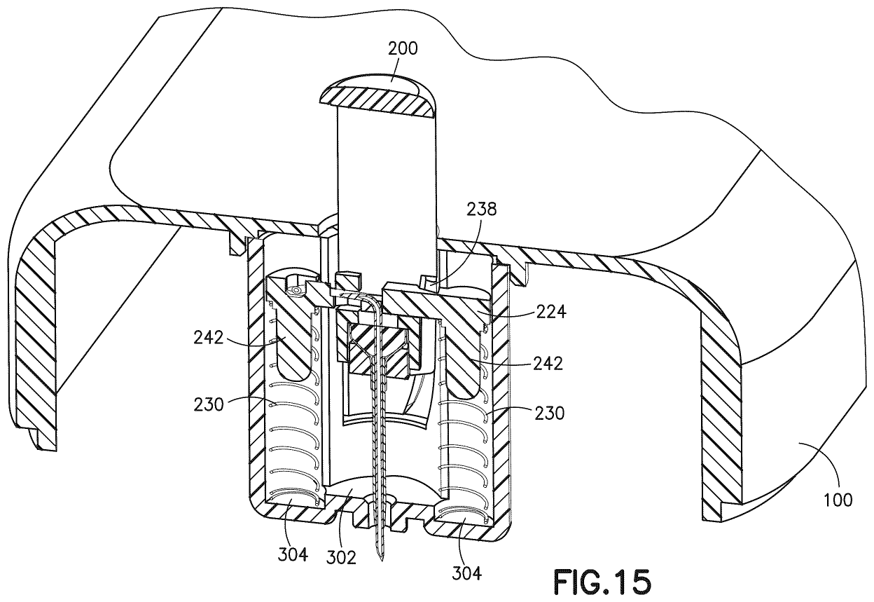

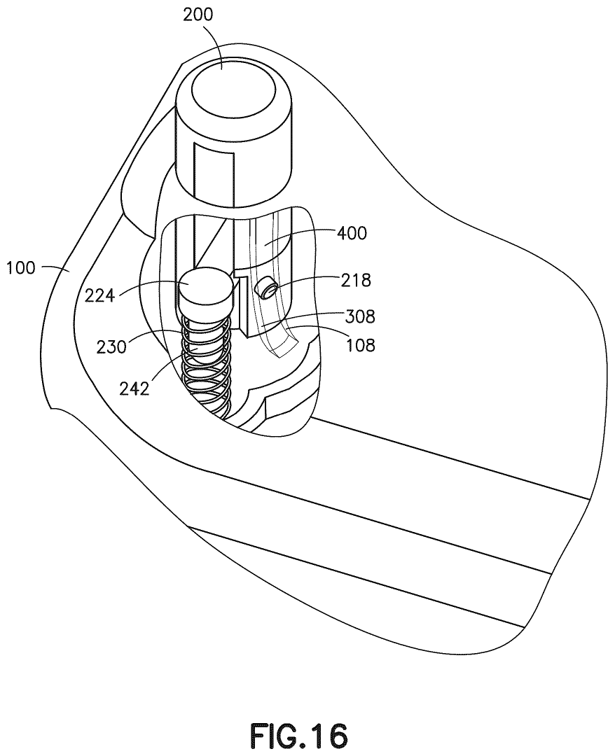

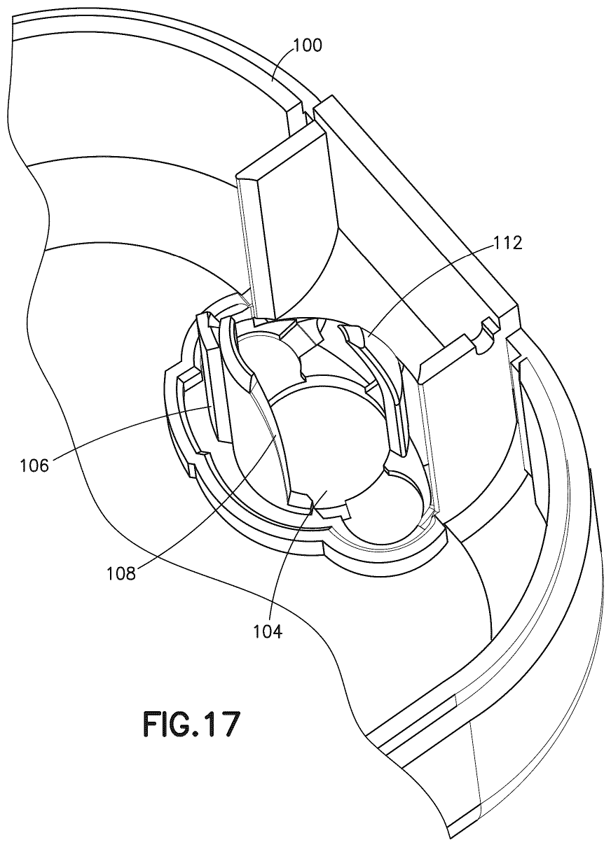

After the release of the button 200, the button subassembly and components therein begin to travel through the mechanism housing 300. FIG. 15 shows a view of the insertion device at the beginning of such insertion. FIG. 15 is a sectional view of the fully assembled insertion device of FIG. 1 in an intermediate activation state in accordance with an embodiment of the present invention.

FIG. 15 also illustrates one of the two teeth 238 on the release collar 208 that couples the introducer needle hub 224 and catheter/septum subassemblies. In this position the teeth 238 are locked over the top the introducer needle hub 224 so as the button 200 is pressed down, the introducer needle bub 224 moves down as well. As the button 200 is pressed down, the introducer needle hub 224 moves down as well, which results in the introducer needle 222 and catheter 202 being simultaneously inserted into a user skin surface (not shown), and also results in the introducer needle hub 224 compressing the springs 230. In order to create an insertion device with a small foot print, each of the springs 230 has a small diameter relative to the compression length which, if unsupported, would cause the springs to buckle during compression. The bosses 242 on the introducer needle hub 224 translate through the middle of the springs 230 during compression to prevent the springs 230 from buckling. In the exemplary embodiment, the springs 230 are compressed, and exert an expansion force to retract the introducer needle bub and introducer needle. However, in this or other embodiments of the present invention, one or more extension springs can be used, and exert a retraction force to retract the introducer needle hub and introducer needle.

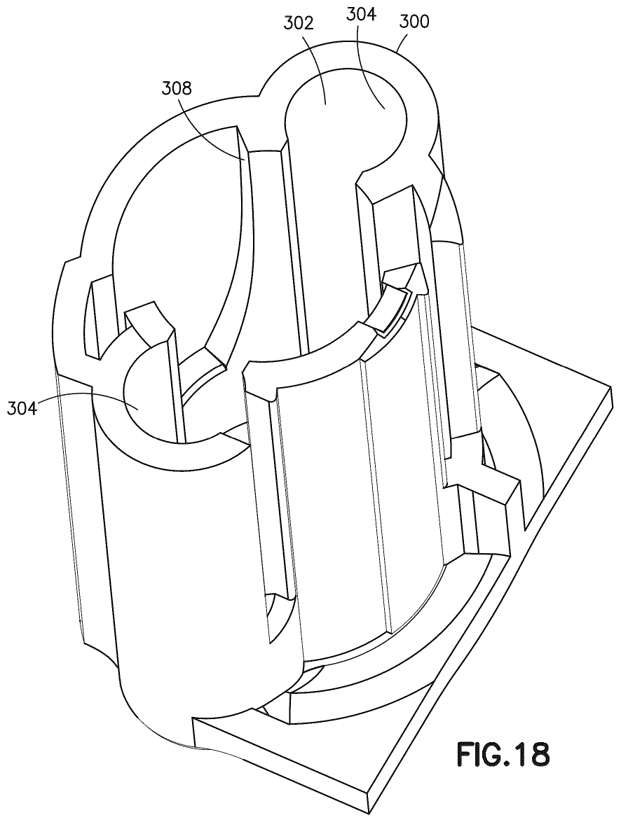

As noted above, the catheter/septum subassembly of FIG. 5 is attached to the button 200 and introducer needle hub 224 but is free to rotate up to 20 degrees around the primary axis. In this case, the primary axis is defined as the axis extending along the geometric center of the insertion needle 222. Slots 216 are provided in the button 200 to allow linear travel of the introducer needle hub 224 relative to the button 200, but prohibit rotational movement of the introducer needle hub relative to the button 200. The slots 216 in the button 200 also allow rotational movement of the radial operation pin 218 of the release collar 208 relative to the button 200. The angle of this rotation is controlled by the radial operation pin 218 extending from the release collar 208. During insertion, that is, downward travel of the button subassembly, the radial operation pin 218 travels in a helical pathway 400 created by the combined features in the top housing 100 and mechanism housing 300. During such travel, the radial operation pin 218 of the release collar 208 rotates the release collar 208 to eventually release the introducer needle subassembly from the catheter/septum subassembly. The surfaces 108 in the top housing 100, and 308 in the mechanism housing 300 that create the helical pathway 400 are divided between two parts. so that both parts can be molded without slides. That is, by creating the helical pathway 400 using the coupling of two separately molded parts, a single part having the slide or pathway molded therein is not required, significantly simplifying the manufacture of the insertion device. FIGS. 17 and 18 show the surface 108 in the top housing 100, and 308 in the mechanism housing 300 that create the helical pathway 400 when assembled.

FIG. 17 is an bottom view of the top housing 100 of the insertion device of FIG. 1 illustrating a portion of the pathway surface, and FIG. 18 is a view of the mechanism housing 300 of the insertion device of FIG. 1 illustrating the remaining portion of the pathway surface of the radial operation pin 218 in accordance with an embodiment of the present invention. As shown in FIG. 17, the projection 106 of the top housing 100, into which the button subassembly is slidably disposed, includes an edge that can be provided with a similar curved, contoured, or otherwise configured shape 108 that, upon assembly with the mechanism housing 300, forms one half, side or portion of the helical pathway 400. As shown in FIG. 18, an inner diameter or chamber surface of the mechanism housing 300, into which the button subassembly is slidably disposed, can be provided with a curved, contoured, or otherwise configured shape 308 that, upon assembly with the top housing 100, also forms one half, side or portion of the helical pathway 400. When the top housing 100 and mechanism housing 300 are assembled, the elements 108 and 308 form the helical pathway 400. The pathway is helical to induce a rotational movement of the release collar 208 relative to the button 200 by guiding the radial operation pin 218 therein, as the button 200 and release collar 208 travel in a linear direction.

As noted above, the slots 216 provided in the button 200 allow movement of the radial operation pin 218 of the release collar 208. Further, the catheter/septum subassembly of FIG. 5 is attached to the button 200 and introducer needle hub 224, but is free to rotate up to 20 degrees around the primary axis. Such 20 degrees of rotation permits the travel of the radial operation pin 218 of the release collar 208 in the helical pathway 400. As the button 200 is pressed down, the release collar 208 and radial operation pin 218 of the release collar 208 move down as well through the stationary top housing 100 and mechanism housing 300. The radial operation pin 218 of the release collar 208 therefore, slidably disposed in the helical pathway 400, rotates the release collar when moved down through the stationary top housing 100 and mechanism housing 300 by the button 200.

In the pre-activation state, the radial operation pin 218 angle is constrained to an orientation in which the teeth 238 of the release collar 208 are fully engaged with the introducer needle hub 224. During button 200 movement between the pre-activation state and the post-activation state, the radial operation pin 218 of the release collar 208 rotates the release collar 208 when moved through helical pathway 400 of the stationary top housing 100 and mechanism housing 300.

In the post-activation state, the radial operation pin 218 has been rotated up to 20 degrees, which decouples the introducer needle hub 224 from the teeth 238 of the release collar 208, freeing the introducer needle hub 224 from the release collar 208, to be retracted by the compressed springs 230. The release collar 208 and other elements of the catheter/septum subassembly are left in the down and inserted position.

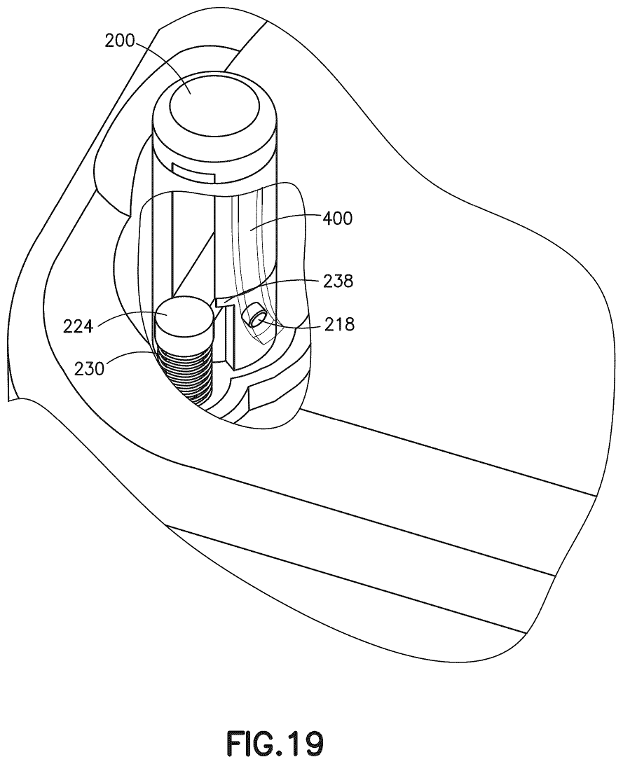

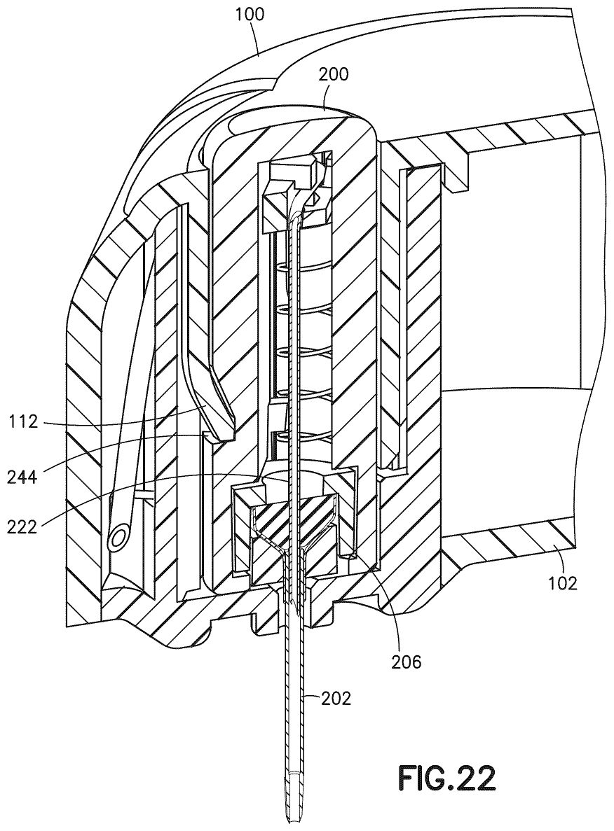

FIG. 19 shows the insertion device during insertion of the introducer needle 222 and catheter 202 and at a point just before the introducer needle hub 224 is released by the radial operation pin 218 of the release collar 208 for retraction. The radial operation pin 218 and the release collar 208 is almost fully rotated by engagement with the helical pathway 400 and where, at the end of rotation by the helical pathway 400, the teeth 238 on the release collar 208 are about to move free of the detents 240 of the introducer needle hub 224 and release the introducer needle hub 224 so it can be pushed up and retracted by the springs 230. That is, as the radial operation pin 218 and the release collar 208 are rotated by engagement with the helical pathway 400, the teeth 238 on the release collar 208 simultaneous rotate until free of the detents 240 of the introducer needle hub 224. At this point, the release collar 208 being held down by the button 200, is no longer secured to the introducer needle hub 224, and the springs 230 force the introducer needle hub 224 and introducer needle 222 upward and into the retracted position, leaving the catheter/septum subassembly in the down and inserted position. The button 200 is locked in the down position, thereby holding the catheter/septum subassembly in the down and inserted position. The lock arm 112 that protrudes from the top housing 100 that retains the button subassembly in place during assembly can also be configured to snap into a detent 244 in the button 200 in the post-activation state locking the button subassembly in place keeping the catheter in the skin as shown in FIG. 22.

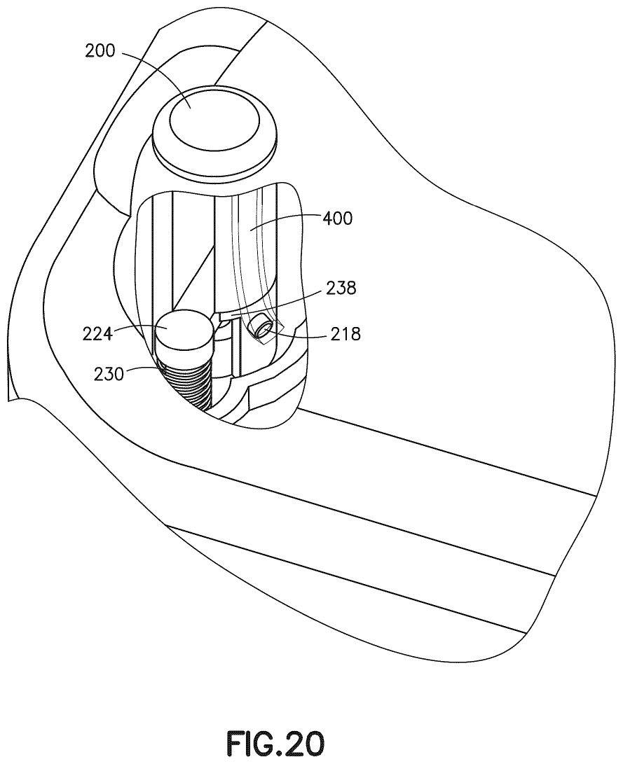

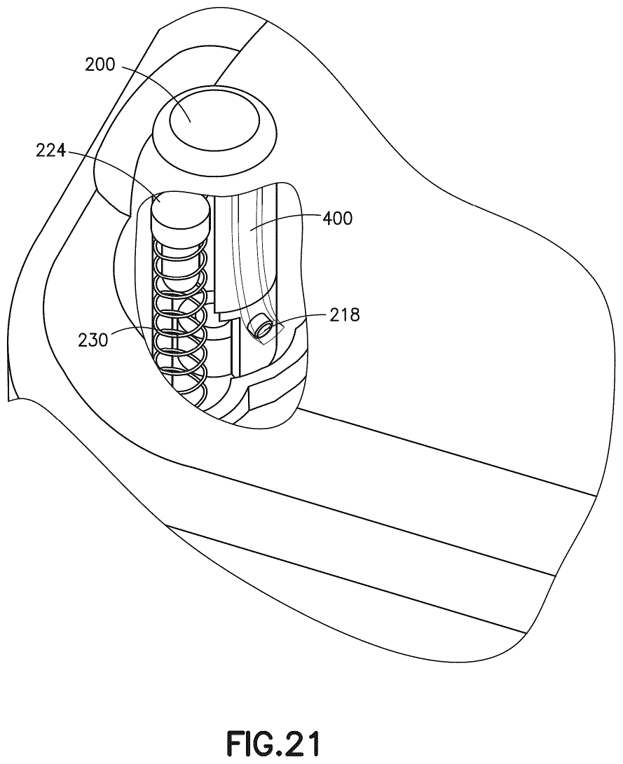

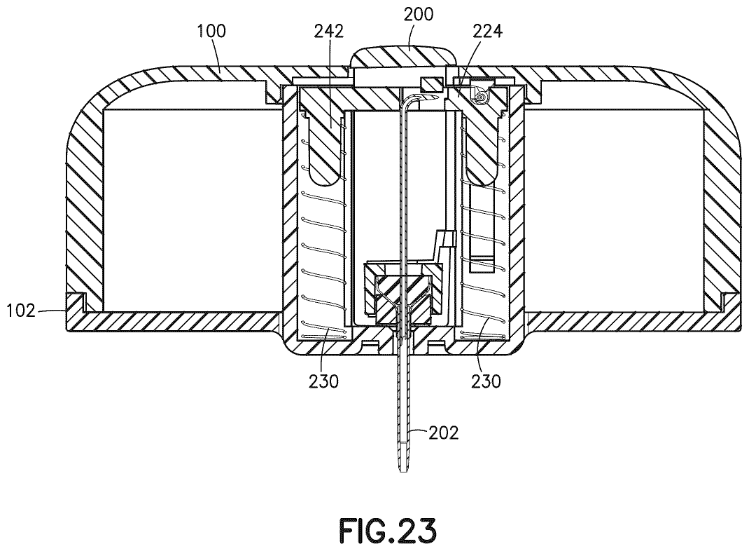

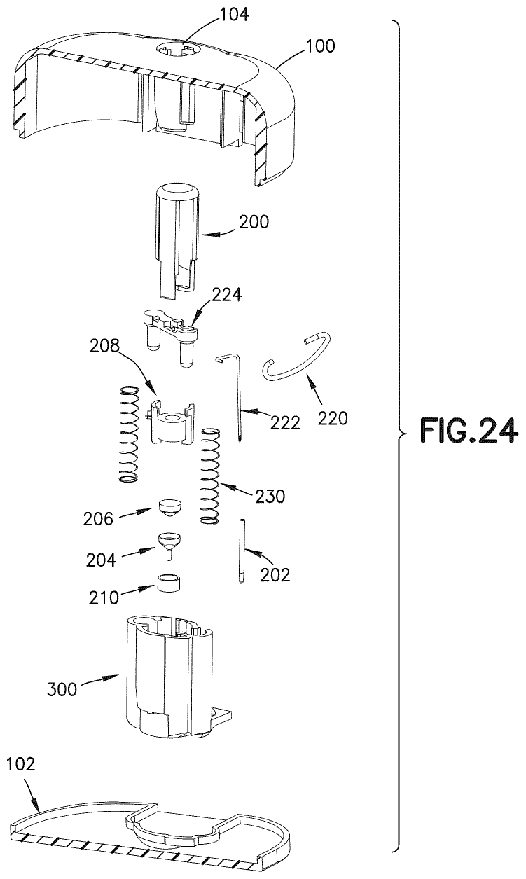

FIG. 20 shows the insertion device just at full insertion of the introducer needle 222 and catheter 202. The retraction springs 230 are fully compressed and the radial operation pin 218 and release collar 208 have been rotated to an extent required for decoupling the teeth 238 of the release collar 208 from the introducer needle hub 224 to release the introducer needle hub 224 for retraction as shown in FIGS. 21 and 23. FIGS. 21 and 23 show the insertion device in a post-activation state. At this point, the release collar 208 being held down by the button 200, is no longer secured to the introducer needle hub 224, and the springs 230 force the introducer needle hub 224 and introducer needle 222 upward and into the retracted position, leaving the catheter/septum subassembly in the down and inserted position.

The introducer needle 222 retracts farther into the housing than its pre-activation state position to ensure needle stick shielding and to protect the catheter from damage. The tip of the introducer needle 222 remains sealed by the septum 206 in the fluid path to form an uninterrupted fluid path with the catheter 202. In this or other embodiments, the tip or distal portion of the introducer needle 222 remains within the catheter 202 and scaled by the septum 206 to form an uninterrupted fluid path with the catheter 202.

In the exemplary embodiments, manual insertion of the introducer needle and catheter allows the insertion device to be smaller, simpler and cheaper than insertion devices employing spring assisted insertion. Other patch pump plastic catheter insertion mechanisms use insertion springs which are large relative to the retraction spring because the insertion force is large relative to the retraction force. Fully integrated, spring assisted insertion also requires angled insertion for a low profile device which increases the stroke and greatly increases the wound and mechanism size. The insertion spring serves no purpose after insertion, but simply takes up room in the device wherein size is one of the most important user requirements for the product.

In the exemplary embodiments, the dual retraction spring configuration also allows for a very small size. One barrel of the insertion device housing guides the button and catheter, and the adjacent barrels house the two retraction springs. Having the springs in separate barrels and directed by bosses on the introducer needle hub allows for much smaller springs than a single barrel configuration in which the spring is coaxial with the catheter. A single coaxial spring creates access to the button assembly since spring design limitations require the spring to extend nearly from the bottom of the housing to the top. Access is required for features like the locking arm and if the features are implemented inside the spring, the entire mechanism must grow to accommodate them increasing the mechanism foot print. Passively locking the catheter down and retracting the introducer needle creates the simplest possible manual insertion user interface for a manual insertion mechanism which is a single button push.

As noted, the retraction springs 230 are minimally loaded before use to ensure that the introducer needle 222 retracts into the device completely. The springs 230 load further during insertion. Providing minimally loaded springs and not fully loaded springs in the insertion device, reduces the risk associated with sterilizing and storing loaded springs and simplifies the design.

To operate the insertion device, the user applies the insertion device to a skin surface using an adhesive upon the base 102 of the device. The user then manually pushes the protruding button 200 until breaking or deforming the ribs 236. The button 200, now suddenly free to travel, is rapidly pushed into the top housing 100 and serves to push and insert the plastic catheter 202 and introducer needle 222 into a user skin surface. As the button 200 is being pushed, the release collar 208 is rotated by the radial operation pin 218 of the release collar 208 moving through helical pathway 400. The release collar 208 is rotated to an extent required for decoupling the release collar 208 from the introducer needle hub 224, and the introducer needle hub 224 and introducer needle 222 are then retracted to a retracted position, exceeding that of the original needle position to ensure needle shielding. The plastic catheter 202 now uncoupled from the introducer needle 222 is left in the down and inserted position. The button 200 automatically locks in the down position, flush with the top of the housing, which also locks the catheter at the desired depth in the subcutaneous layer. A sensor (not shown) can be provided to sense the post-activation state and advise other electronics (not shown) that the catheter has been inserted properly which allows the patient to infuse medicament. A pump or reservoir then infuses medicament through the introducer needle, into the catheter and out into the patient's subcutaneous layer.

To best target the desired depth, the base can include skin interface geometry to achieve and maintain a desired insertion depth, avoid skin surface tenting, and/or tension the skin surface at the insertion site. FIGS. 48-50 show examples of such skin interface geometry with a catheter deployed. In the perspective view of the device 502, a post 504 from which the catheter 506 extends during placement, protrudes into the skin surface (not shown) which helps prevent shallow catheter tip insertion in cases where the skin tented. The post 504 can extend from the base surface of the device 502 to any desired length, and can be rounded and/or chamfered at the distal end contacting the skin surface.

A well 508 can be provided surrounding the post 504. The well 508 provides space for the skin that is displaced during insertion and helps the post 504 protrude into the skin surface. A wall 510 surrounds and defines the well 508, and can extend from the base surface of the device 502 to any desired length and can be rounded and/or chamfered at the distal end contacting the skin surface. The round opposing cylinders 512 in FIGS. 48-50 can be provided, or excluded from the geometry as desired.

In the above exemplary embodiment, the insertion mechanism can be created as a subassembly in the top housing 100. This allows the insertion mechanism to be handled easily during production so the other sub systems can be assembled. Alternatively, the insertion mechanism can be created as a subassembly separate from the top housing 100 or base 102 as shown in FIG. 25, or as a subassembly in the base 102 as shown in FIG. 26.

In FIG. 25, a completed button subassembly 250, substantially the same as described in regard to FIG. 9, is secured within a mechanism housing 350, substantially the same as described in regard to FIG. 4, using, for example, snaps or detents 252. In this case, the insertion mechanism is created as a subassembly separate from the top housing 100 or base 102. Upon completion, the insertion mechanism of FIG. 25 can then be assembled with one or more of the top housing 100 and base 102.

In FIG. 26, a completed button subassembly 260, substantially the same as described in regard to FIG. 9, is secured within a mechanism housing 360, substantially the same as described in regard to FIG. 4. In this case, the insertion mechanism is created as a subassembly in the base 102. Further, in each embodiment of FIGS. 25 and 26, the surfaces that create the helical pathway as described above in regard to FIGS. 17 and 18, can be provided in the button subassembly 250 and mechanism housing 350, and in the button subassembly 260, mechanism housing 360 and/or base 102, such that the surfaces can again be divided between two parts, so that both parts can be molded without slides.

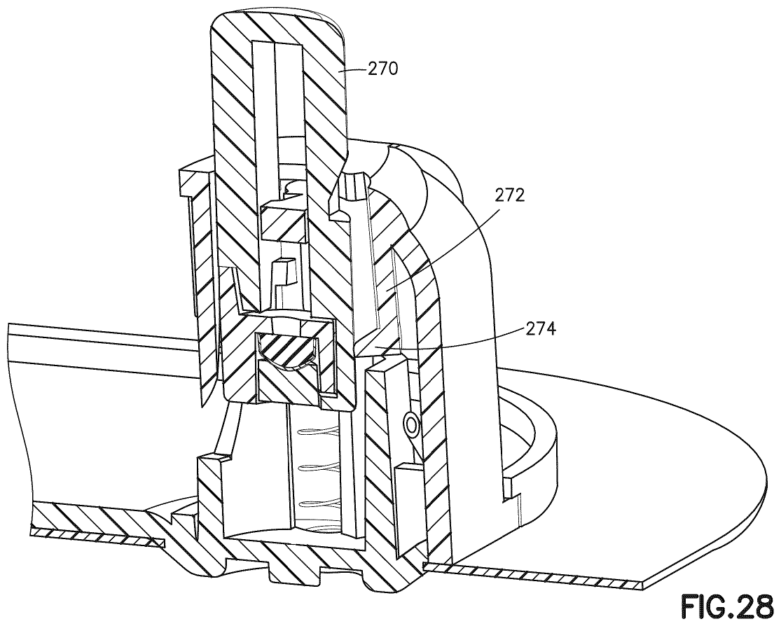

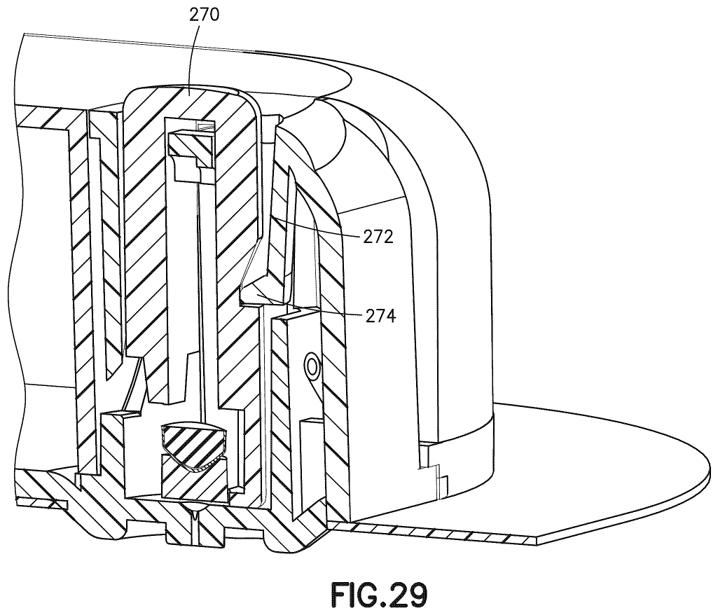

In the above exemplary embodiment, the ribs 236 determine the minimum insertion force to start activation of the device which ensures full activation. Alternatively, the lock arm 112 can be configured to also determine the minimum activation force. As noted above, the lock arm 112 protrudes from the top housing and snaps into a detent in the button in the post-activation state locking the button subassembly in place keeping the catheter in the skin. FIG. 27 shows another embodiment of the lock arm 272 including a flange 274 on the lock arm that holds the button 270 in the pre-activation position. The contoured flange 274 of the lock arm 272 protrudes and captures a bottom edge of the button 270 in the pre-activation state, holding the button subassembly in place until a sufficient force is applied to the button. Once a sufficient force is applied to the button 270, the flange 274 is deflected clear of the button 270. The lock arm 272 bends out of the path of the insertion button 270 when sufficient force is applied. FIG. 28 shows the device in an intermediate state during insertion. The lock arm 272 would be bent outward instead of interfering as FIG. 28 depicts. The minimum deflection force of the lock arm 272 and flange 274 ensures that the user pushes hard enough to fully insert the catheter. The lock arm 272 and flange 274 then snap into the detent 276 in the button 270 when the button reaches the down most position which locks the button and catheter as shown in FIG. 29.

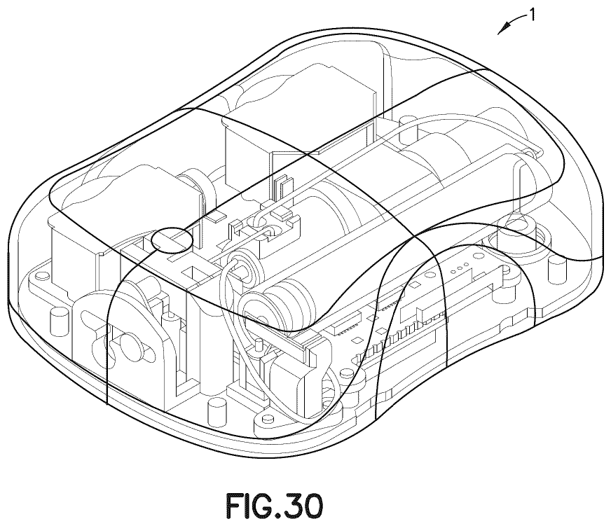

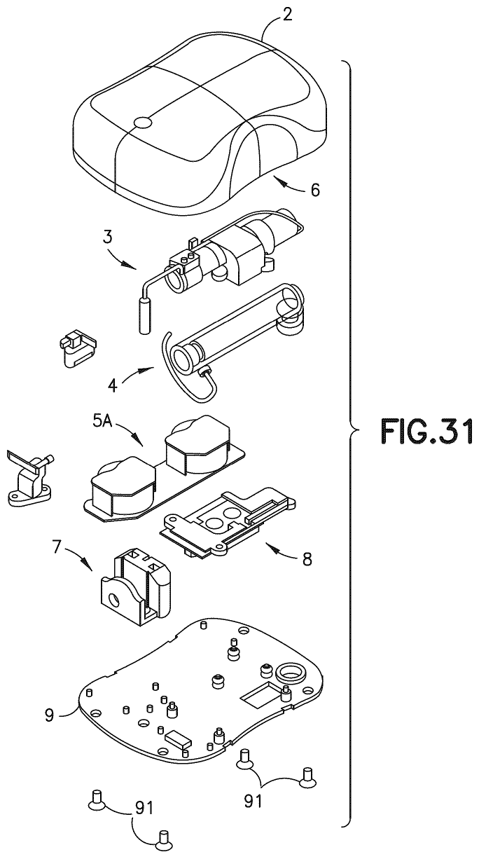

In the above embodiments, a patch pump can be provided with one or more of the described features. FIG. 30 is a perspective view of an exemplary embodiment of a patch pump 1 according to an exemplary embodiment of the invention. The patch pump 1 is illustrated with a see-through cover for clarity and illustrates various components that are assembled to form the patch pump 1. FIG. 31 is a view of the various components of the patch pump of FIG. 30, illustrated with a solid cover 2. The various components of the patch pump 1 may include: a reservoir 4 for storing insulin; a pump 3 for pumping insulin out of the reservoir 4; a power source 5 in the form of one or more batteries; an insertion mechanism 7 for inserting an inserter needle with a catheter into a user's skin; control electronics 8 in the form of a circuit board with optional communications capabilities to outside devices such as a remote controller and computer, including a smart phone; a dose button 6 on the cover 2 for actuating an insulin dose, including a bolus dose; and a base 9 to which various components above may be attached via fasteners 91. The patch pump 1 also includes various fluid connector lines that transfer insulin pumped out of the reservoir 4 to the infusion site.

As noted above, it should be understood that inserter mechanisms come in various configurations. In some embodiments, the inserter mechanism inserts a soft catheter into the skin. In these embodiments, typically the soft catheter is supported on a rigid insertion needle. The insertion needle is inserted into the skin along with the soft catheter, and then retracted from the skin, leaving the soft catheter in the skin. In other embodiments, a soft catheter is not provided, and the insertion needle remains in the skin and forms a portion of the insulin flow path to deliver insulin until the infusion is finished. Insertion needles are typically hollow, and need to be hollow if they form part of the insulin flow path. However, insertion needles that support a soft catheter and then retract may be solid or hollow. If the insertion needle deploys a soft catheter, and retracts but remains part of the insulin flow path, then the insertion needle should be hollow. However, if the insertion needle deploys a soft catheter and then retracts but does not form part of the insulin flow path, then the insertion needle may be solid or hollow. In either case, the insertion needle is preferably rigid enough to reliably penetrate the skin, but otherwise may be made flexible enough to provide comfort to the user.

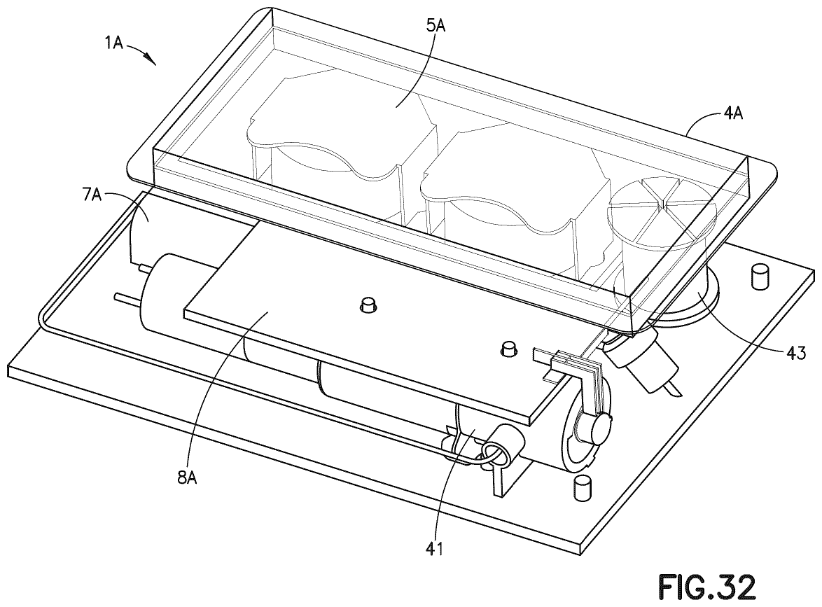

FIG. 32 is a perspective view of an alternative design for a patch pump 1A having a flexible reservoir 4A, and illustrated without a cover. Such arrangement may further reduce the external dimensions of the patch pump 1A, with the flexible reservoir 4A filling voids within the patch pump 1A. The patch pump 1A is illustrated with a conventional cannula insertion device 7A that inserts the cannula, typically at an acute angle, less than 90 degrees, at the surface of a user's skin. The patch pump 1A further comprises: a power source 5A in the form of batteries; a metering sub-system 41 that monitors the volume of insulin and includes a low volume detecting ability; control electronics 8A for controlling the components of the device; and a reservoir fill port 43 for receiving a refill syringe 45 to fill the reservoir 4A.

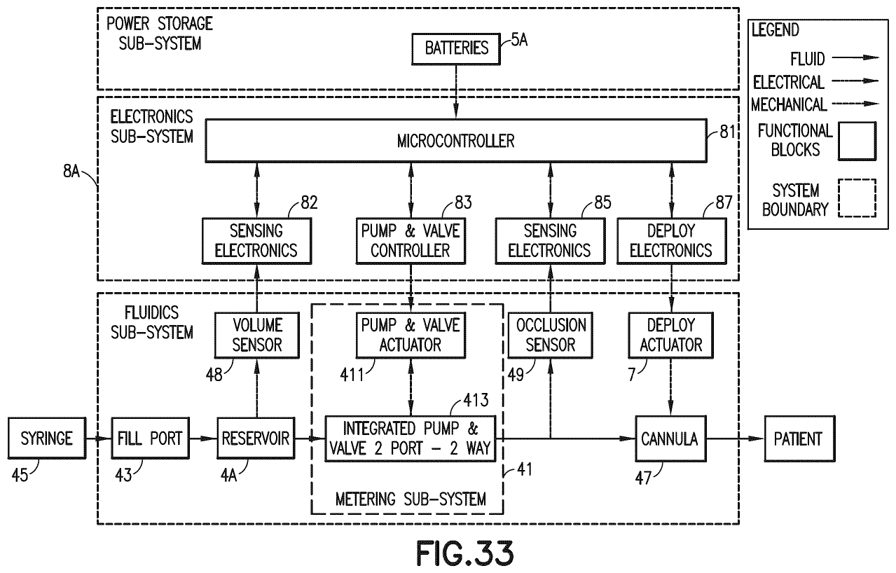

FIG. 33 is a patch-pump fluidic architecture and metering sub-system diagram of the patch pump 1A of FIG. 32. The power storage sub-system for the patch pump 1A includes batteries 5A. The control electronics 8A of the patch pump 1A may include a microcontroller 81, sensing electronics 82, pump and valve controller 83, sensing electronics 85, and deployment electronics 87 that control the actuation of the patch pump 1A. The patch pump 1A includes a fluidics sub-system that may include a reservoir 4A, volume sensor 48 for the reservoir 4A, a reservoir fill port 43 for receiving a refill syringe 45 to refill the reservoir 4A. The fluidics sub-system may include a metering system comprising a pump and valve actuator 411 and an integrated pump and valve mechanism 413. The fluidics sub-system may further include an occlusion sensor, a deploy actuator, as well as the cannula 47 for insertion into an infusion site on the user's skin. The architecture for the patch pumps of FIGS. 30 and 31 is the same or similar to that which is illustrated in FIG. 33.

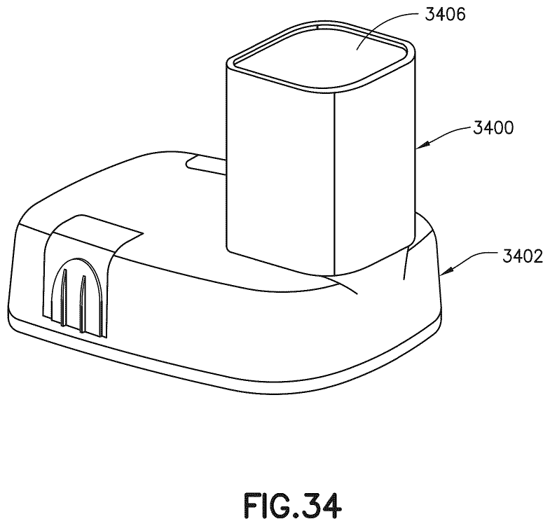

Another exemplary embodiment of the insertion mechanism is illustrated at FIG. 34. This embodiment includes an external applicator 3400 that attaches to the IDD 3402. Also included is an internal module 3404 that is installed inside the housing of the IDD 3402. As shown, and will be described in further detail below, the external applicator 3400 is attached to the IDD 3402, in order to interact with the internal module 3404, and automatically releases from the IDD 3402 when cannula insertion is complete. Cannula insertion is initiated by a user pressing a release button 3406 of the external applicator 3400.

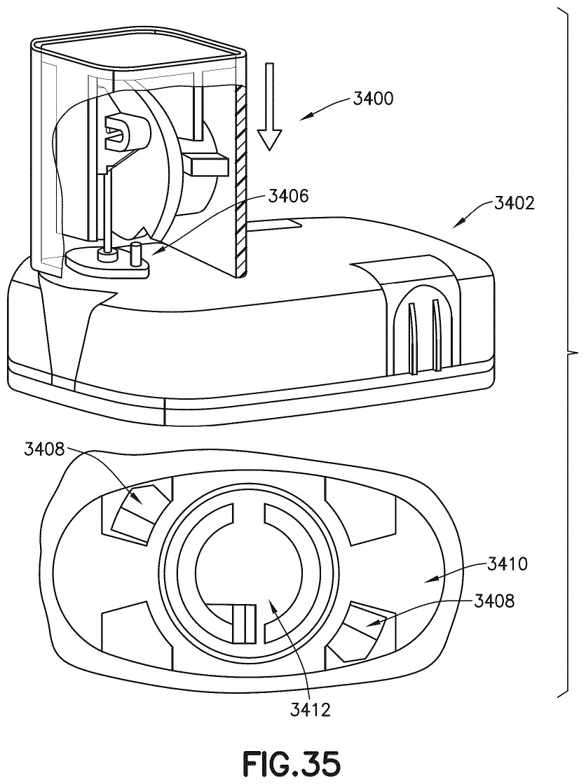

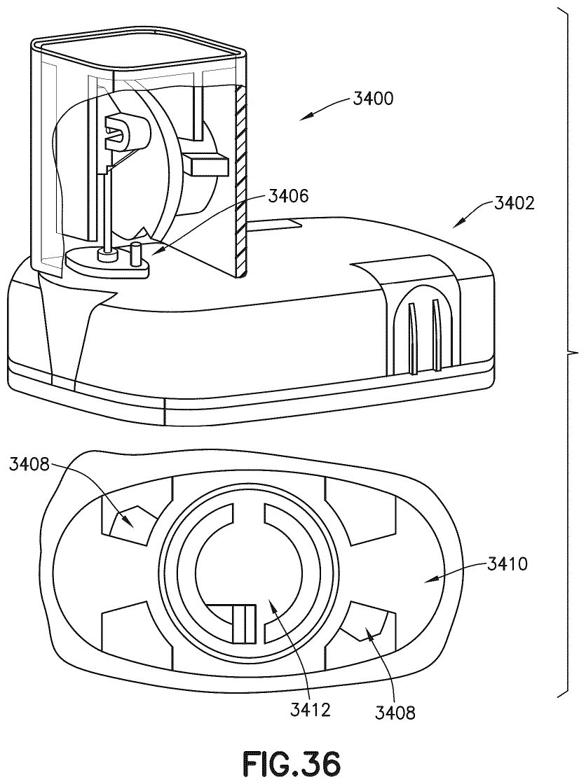

As illustrated in FIG. 35, prior to use the external applicator 3400 and internal module 3404 subassemblies are assembled. The external applicator 3400 is pressed onto the IDD 3402 in the direction of the arrow shown in FIG. 35. A passive release 3406 of the external applicator 3400 includes locking features 3408 as shown in the top-down view in FIG. 35. As the external applicator 3400 is assembled to the IDD 3402, the passive release rotates counter clockwise, as shown in FIG. 36, and the locking features 3408 lock onto a cap 3410 of the internal module 3404. The rotation is preferably cause by interaction of a feature on the passive release and the cap 3410 as illustrated by the arrow in FIG. 36.

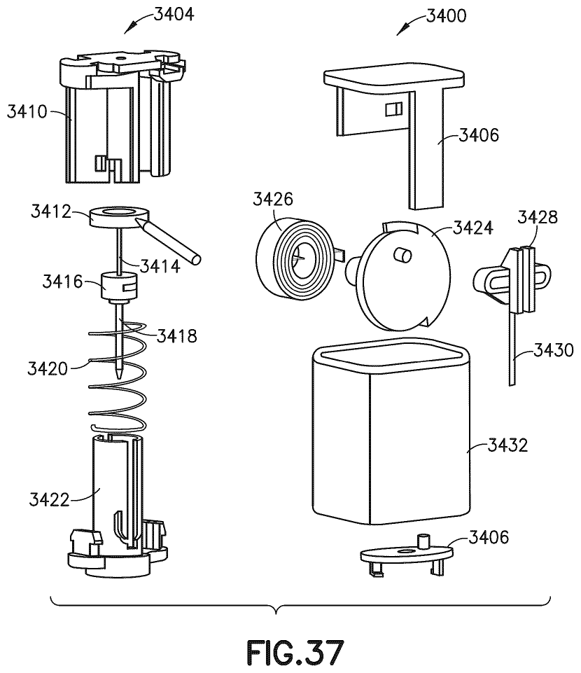

FIG. 37 is an exploded view of the internal module 3404 subassembly and the external applicator 3400 subassembly. The components of each will now be briefly described. Internal module 3404 includes cap 3410, needle hub 3412 and needle 3414, catheter hub 3416 and catheter 3418, return spring 3420 and base 3422. External applicator 3400 includes release button 3406 drive disc 3424, clock spring 3426, yoke 3428, drive pin 3430, applicator housing 3432 and passive release 3406.

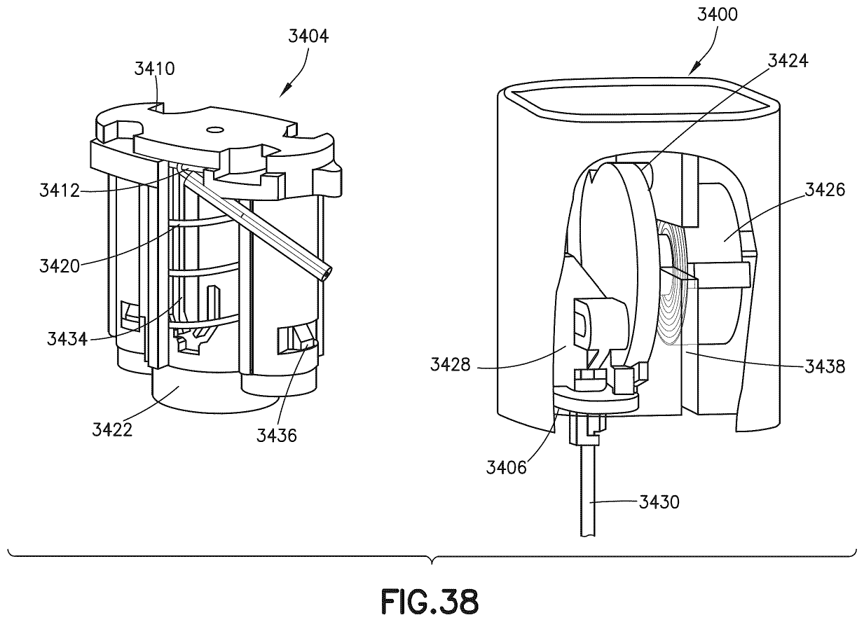

FIG. 38 is an assembled isometric view of internal module 3404 and external applicator 3400. As illustrated, base 3422 includes flex arms 3434 which permit catheter hub 3416 to pass, and then retain the catheter hub 3416 in the deployed position, as will be described in further detail below. Base 3422 also includes snaps 3436 which engage corresponding features in the cap 3410 during assembly, and lock the cap 3410 to the base 3422. In the external applicator 3400, drive wheel 3424 and clock spring 3426 are supported on support features 3438 molded into the applicator housing 3432

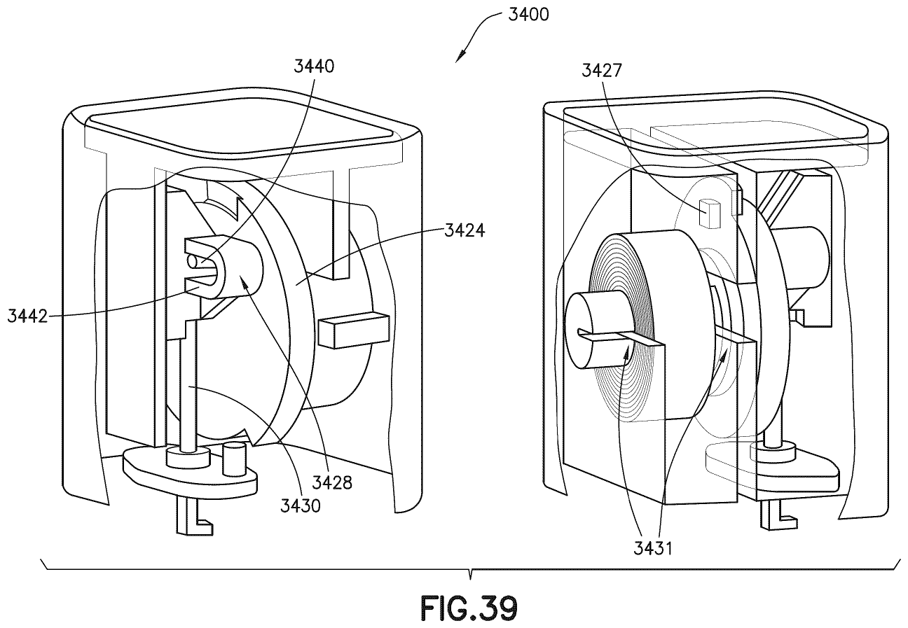

FIG. 39 illustrates the assembled external applicator 3400 from first and opposite sides in a cutaway view. As will be described in further detail below, the drive disc 3424 includes a drive post 3440 that is received in a slot 3442 of the yoke 3428. These interact to convert rotation of the drive disc 3424 into a down and up reciprocation of the yoke 3428 and drive pin 3430. Applicator housing 3432 is manufactured with support features to support the clock spring 3426 both radially and axially, as shown at 3431. Release button 3406 includes a release feature 3427 that retains the drive disc 3424 until the button is presses, disengaging the drive disc 3424 from the release button 3406, and permitting the clock spring 3426 to rotate the drive disc 3424. Applicator housing 3432 also includes ridges 3442 which support the yoke 3428 and otherwise assists to keep the assembled parts in place.

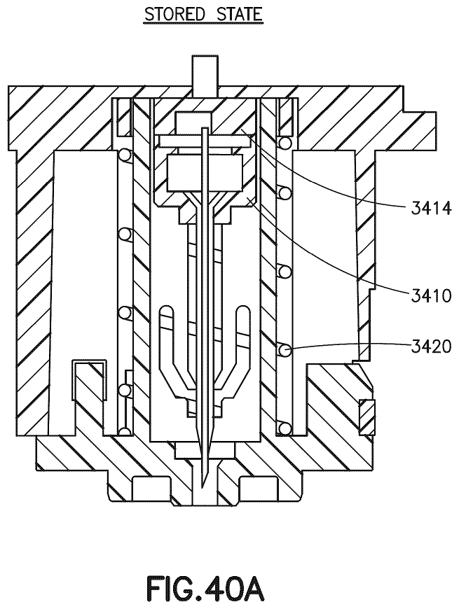

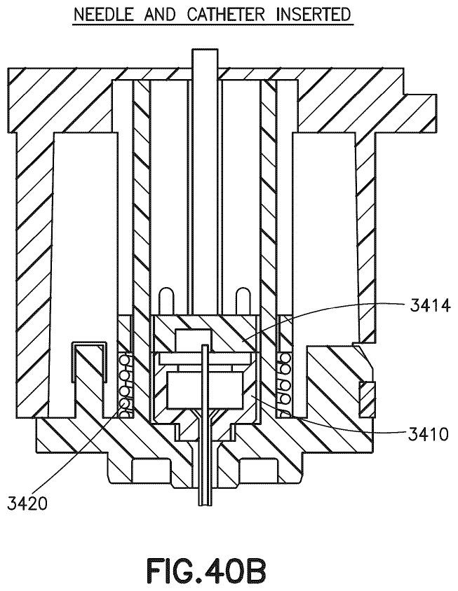

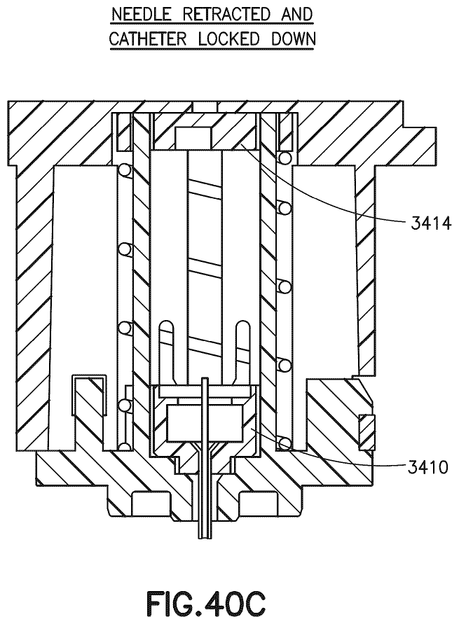

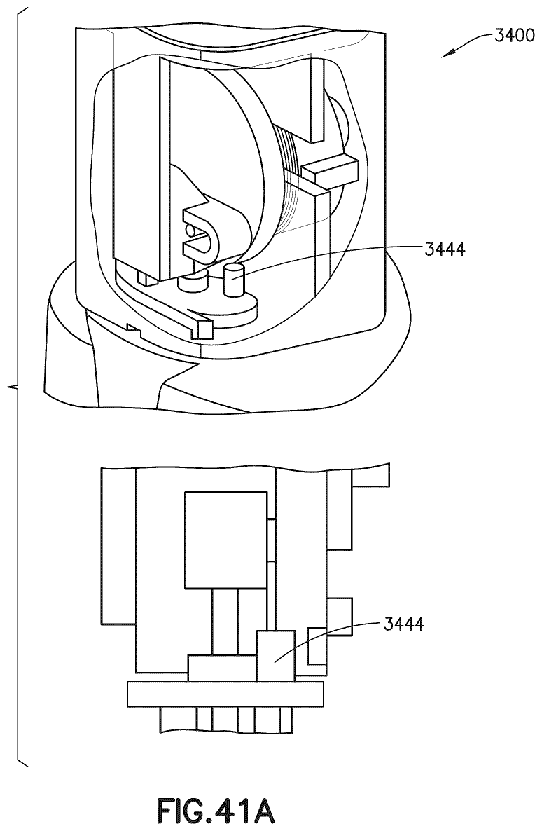

FIGS. 40A-40C illustrate the process of needle insertion once the release button is pressed. FIG. 40A illustrates the stored state. Needle hub 3412, catheter hub 3416 and drive pin 3430 are in the upward position, and return spring 3420 is in an uncompressed state. As the dive disc 3424 rotates, drive pin 3430 and yoke slot 3442 interact to drive the yoke 3428 and drive pin 3430 downward. The fully inserted position is illustrated in FIG. 40B, when the drive disc 3424 has rotated approximately 180 degrees. Of note in FIG. 40A are flex arms 3434 which flex and permit the catheter hub 3416 to pass and be captured in the downward position. Return spring 3420 is compressed by the needle hub 3412. As drive disc 3424 continues to rotate the yoke 3428 reciprocates upward and pulls needle hub 3412 upward to the upper position shown in FIG. 40C, driven by return spring 3420 as the drive disc 3424 continues to rotate and permits the yoke 3428 to return to the upper position.

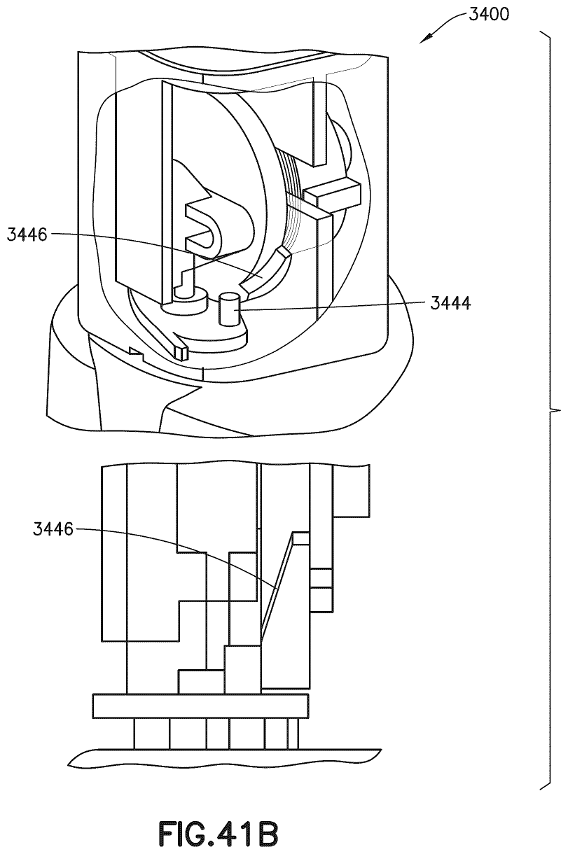

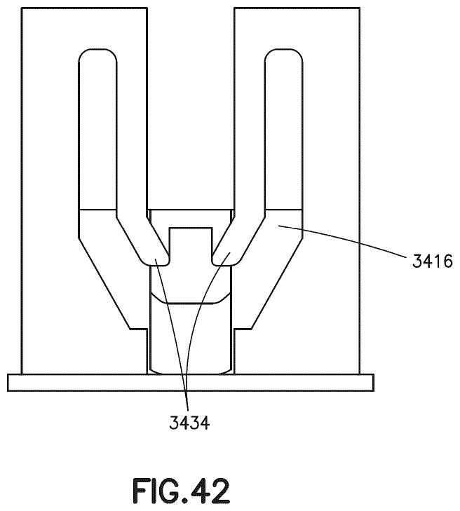

FIGS. 41A and 41B illustrate how the external applicator 3400 is automatically released from the IDD 3402. As shown in FIG. 41A, passive release 3406 includes a post 3444. Drive disc 3424 includes a cam surface 3446 shown in FIG. 41B. Rotation of the drive disc 3424 causes the cam surface 3446 to push the post 3444, rotating the passive release in a clockwise direction, and thus disengaging the external applicator 3400 from the IDD 3402. FIG. 42 illustrates the flex arms 3434 after the catheter hub 3416 has moved downward passed the terminal end of the flex arms 3434, such that the catheter hub 3416 becomes captured in the downward position.

Although only a few exemplary embodiments of the present invention have been described in detail above, those skilled in the art will readily appreciate that many modifications are possible in the exemplary embodiments without materially departing from the novel teachings and advantages of this invention. Accordingly, all such modifications are intended to be included within the scope of the appended claims and their equivalents.

1. A catheter insertion device, comprising:
an internal module subassembly and an external applicator subassembly, wherein the internal module subassembly is housed within a housing of a medicine delivery device and the external applicator subassembly is removably attached to the medicine delivery device;
wherein the internal module subassembly comprises a needle hub with an insertion needle fixed thereto, a cap with a hole therein, a base that snaps to the cap, a return spring between the needle hub and the base, and a catheter hub with a catheter fixed thereto;
wherein the external applicator assembly comprises a release button, a housing and a passive release rotatable between a first orientation that is released from the medicine delivery device and a second position that is fixed to the medicine delivery device; the external applicator subassembly further comprising a drive disc and clock spring, and a yoke fixed to t drive pin, wherein when the button releases the drive disc, the drive disc causes the yoke and drive pin to reciprocate such that the drive pin drives the needle hub downward to insert the catheter, and then the drive pin returns to the external applicator housing.

2. The catheter insertion device of claim 1, wherein the drive disc comprises a cam surface that orients the passive release in the second position after the release button is pressed.

3. The catheter insertion device of claim 1, wherein the yoke comprises a slot and the drive disc has a drive post inserted in the slot to convert rotational motion of the drive disc into reciprocal linear motion of the yoke and drive pin.

4. The catheter insertion device of claim 1, wherein the internal module subassembly comprises flex arms that permit the catheter bub to pass distal ends of the flex arms to reach a downward position of the catheter hub, and lock the catheter hub in the downward position.

5. The catheter insertion device of claim 1, wherein the release button comprises a release feature that prevents the disc drive from rotating prior to the release button being pressed, and which releases the drive disc when the release button is pressed.
